SEQUENCE LISTING (286 sequences)

SEQ ID NO: 1 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 2 - protein, 479 aa, Pseudomonas fluorescens
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 3 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacctgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 4 - protein, 479 aa, synthetic construct
MSLLIRGATIVTHDESYRADVYCADGVIKAIGENLDIPAGAEVLDGSGQYLMPGGIDPHTHLQLPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 5 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatggagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 6 - protein, 479 aa, synthetic construct
MSLLIRGATIVTHDESYRADVYCADGVIKAIGENLDIPAGAEVLDGSGQYLMPGGIDPHTHMELPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 7 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagattccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 8 - protein, 479 aa, synthetic construct
MSLLIRGATIVTHDESYRADVYCADGVIKAIGENLDIPAGAEVLDGSGQYLMPGGIDPHTHMQIPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 9 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 10 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 11 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagtctccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 12 - protein, 479 aa, synthetic construct
MSLLIRGATIVTHDESYRADVYCADGVIKAIGENLDIPAGAEVLDGSGQYLMPGGIDPHTHMQSPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 13 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcaggcaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 14 - protein, 479 aa, synthetic construct
MSLLIRGATIVTHDESYRADVYCADGVIKAIGENLDIPAGAEVLDGSGQYLMPGGIDPHTHMQAPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 15 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtatatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 16 - protein, 479 aa, synthetic construct
MSLLIRGATIVTHDESYRADVYCADGVIKAIGENLDIPAGAEVLDGSGQYLMPGGIDPHTHMQLPYMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 17 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgctgatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 18 - protein, 479 aa, synthetic construct
MSLLIRGATIVTHDESYRADVYCADGVIKAIGENLDIPAGAEVLDGSGQYLMPGGIDPHTHMQLPLMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 19 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgttttggggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 20 - protein, 479 aa, synthetic construct
MSLLIRGATIVTHDESYRADVYCADGVIKAIGENLDIPAGAEVLDGSGQYLMPGGIDPHTHMQLPFWGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 21 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgttttatggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 22 - protein, 479 aa, synthetic construct
MSLLIRGATIVTHDESYRADVYCADGVIKAIGENLDIPAGAEVLDGSGQYLMPGGIDPHTHMQLPFYGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 23 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgttttttggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 24 - protein, 479 aa, synthetic construct
MSLLIRGATIVTHDESYRADVYCADGVIKAIGENLDIPAGAEVLDGSGQYLMPGGIDPHTHMQLPFFGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 25 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttacgagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 26 - protein, 479 aa, synthetic construct
MSLLIRGATIVTHDESYRADVYCADGVIKAIGENLDIPAGAEVLDGSGQYLMPGGIDPHTHMQLPFMGTVTSEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 27 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgggtccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 28 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPGPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 29 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccggatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 30 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPDPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 31 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgttgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 32 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPLPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 33 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcccttatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 34 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPNPQQSLLEAFHQWRGWAEKSASDYGFHVAITWWSEQVREEMAELVSHHGINSFKHFMAYKNALMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 35 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctttatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 36 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 37 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgttcttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 38 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCSCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 39 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtctttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 40 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCLCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 41 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattatgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 42 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIMNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 43 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattcttaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 44 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKILNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 45 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattcagaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 46 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIQNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 47 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagttaccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 48 - protein, 479 aa, synthetic construct
MSLLIRGATIVTHDESYRADVYCADGVIKAIGENLDIPAGAEVLDGSGQYLMPGGIDPHTHMQLPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 49 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctatatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 50 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAYMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 51 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctttatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 52 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 53 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 54 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNALMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 55 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaaattgtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 56 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGEIVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 57 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaagttgtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 58 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGEVVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 59 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagcatattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 60 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQHITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 61 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtgctgtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 62 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 63 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 64 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSPVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 65 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtcagaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 66 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARQKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 67 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtctgaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 68 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARLKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 69 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtaataaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 70 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARNKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 71 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtgggaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 72 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARGKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 73 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgttttaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 74 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARFKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 75 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagttttggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 76 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSFGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 77 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagttatggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 78 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSYGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 79 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtcatggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 80 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSHGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 81 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccctagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaatggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 82 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSNGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 83 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 84 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGWPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 85 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtgcggatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 86 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGADDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 87 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcttgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 88 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGLDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 89 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggttatgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 90 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGYDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 91 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaatggaccgcagttaaacgt

SEQ ID NO: 92 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKWTAVKR

SEQ ID NO: 93 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgacccctgttaaacgt

SEQ ID NO: 94 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTPVKR

SEQ ID NO: 95 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacag

SEQ ID NO: 96 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKQ

SEQ ID NO: 97 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaactt

SEQ ID NO: 98 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKL

SEQ ID NO: 99 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacct

SEQ ID NO: 100 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKP

SEQ ID NO: 101 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 102 - protein, 479 aa, synthetic construct
MSLLIRGATIVTHDESYRADVYCADGVIKAIGENLDIPAGAEVLDGSGQYLMPGGIDPHTHMQTPFMGTVTSEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 103 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtacatgggcaccgttgcaagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 104 - protein, 479 aa, synthetic construct
MSLLIRGATIVTHDESYRADVYCADGVIKAIGENLDIPAGAEVLDGSGQYLMPGGIDPHTHMQTPYMGTVASEDFYSGTAAGLAGGTTSIIDFVIPLPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 105 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 106 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPLPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 107 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttttcggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 108 - protein, 479 aa, synthetic construct
MSLLIRGATIVTHDESYRADVYCADGVIKAIGENLDIPAGAEVLDGSGQYLMPGGIDPHTHMQTPFFGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 109 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 110 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 111 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattatatggcctataaaaatgcctttatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 112 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHYMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 113 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtggttccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctttatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 114 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVVPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 115 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaaacctgcccgggtcatctgctgctggatgatagcgtgtatcagcatccggattggcagaccgcagcaggctatgtttgctctccgccgcttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 116 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGETCPGHLLLDDSVYQHPDWQTAAGYVCSPPLRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 117 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagtttgcccgcagtatctgctgctggatgatagcgtgtatcagcatccggattggcagaccgcagcaggctatgtttgctctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 118 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVCPQYLLLDDSVYQHPDWQTAAGYVCSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 119 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaaaccctgccgggttatctgctgctggatgatagcgtgtatcagcatccggattggcagaccgcagcaggctatgttatgtctccgccgcttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 120 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGETLPGYLLLDDSVYQHPDWQTAAGYVMSPPLRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 121 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatgttatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 122 - protein, 479 aa, synthetic construct
MSLLIRGATIVTHDESYRADVYCADGVIKAIGENLDIPAGAEVLDGSGQYVMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 123 - DNA, 1437 bp, synthetic construct
  source 1..1437: mol_type = other DNA
atgagcttactgattcgcggcggcaccattgtgacccatgatgaatcttatcgcgccgatgtgtattgtgccgatggtgttattaaagcaattggtgaaaatctggatattccggccggcgcagaagtgttagatgcctcaggccagtatgttatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 124 - protein, 479 aa, synthetic construct
MSLLIRGGTIVTHDESYRADVYCADGVIKAIGENLDIPAGAEVLDASGQYVMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 125 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 126 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 127 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatattatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 128 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 129 - DNA, 1437 bp, synthetic construct
  source 1..1437: mol_type = other DNA
atgagcttactgattcgcggcggcaccattgtgacccatgatgaatcttatcgcgccgatgtgtattgtgccgatggtgttattaaagcaattggtgaaaatctggatattccggccggcgcagaagtgttagatggctcaggccagtatattatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgcgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 130 - protein, 479 aa, synthetic construct
MSLLIRGGTIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 131 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatgcctcaggccagtatattatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 132 - protein, 479 aa, synthetic construct
MSLLIRGATIVTHDESYRADVYCADGVIKAIGENLDIPAGAEVLDASGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 133 - DNA, 1437 bp, synthetic construct
  source 1..1437: mol_type = other DNA
atgagcttactgattcgcggcggcaccattgtgacccatgatgaatcttatcgcgccgatgtgtattgtgccgatggtgttattaaagcaattggtgaaaatctggatattccggccggcgcagaagtgttagatgcctcaggccagtatgttatgccgggcggcattgatccgcatacccacatgcagacgccgtttatgggcaccgttgcaagtgatgatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgcgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 134 - protein, 479 aa, synthetic construct
MSLLIRGGTIVTHDESYRADVYCADGVIKAIGENLDIPAGAEVLDASGQYVMPGGIDPHTHMQTPFMGTVASDDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 135 - DNA, 1437 bp, synthetic construct
  source 1..1437: mol_type = other DNA
atgagcttactgattcgcggcggcaccattgtgacccatgatgaatcttatcgcgccgatgtgtattgtgccgatggtgttattaaagcaattggtgaaaatctggatattccggccggcgcagaagtgttagatggctcaggccagtatcttatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 136 - protein, 479 aa, synthetic construct
MSLLIRGGTIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 137 - DNA, 1437 bp, synthetic construct
  source 1..1437: mol_type = other DNA
atgagcttactgattcgcggcggcaccattgtgacccatgatgaatcttatcgcgccgatgtgtattgtgccgatggtgttattaaagcaattggtgaaaatctggatattccggccggcgcagaagtgttagatgcctcaggccagtatgttatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgcgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 138 - protein, 479 aa, synthetic construct
MSLLIRGGTIVTHDESYRADVYCADGVIKAIGENLDIPAGAEVLDASGQYVMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 139 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatgcctcaggccagtatcttatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 140 - protein, 479 aa, synthetic construct
MSLLIRGATIVTHDESYRADVYCADGVIKAIGENLDIPAGAEVLDASGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 141 - DNA, 1437 bp, synthetic construct
  source 1..1437: mol_type = other DNA
atgagcttactgattcgcggcggcaccattgtgacccatgatgaatcttatcgcgccgatgtgtattgtgccgatggtgttattaaagcaattggtgaaaatctggatattccggccggcgcagaagtgttagatgcctcaggccagtatattatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 142 - protein, 479 aa, synthetic construct
MSLLIRGGTIVTHDESYRADVYCADGVIKAIGENLDIPAGAEVLDASGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 143 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatgcctcaggccagtatgttatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 144 - protein, 479 aa, synthetic construct
MSLLIRGATIVTHDESYRADVYCADGVIKAIGENLDIPAGAEVLDASGQYVMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 145 - DNA, 1437 bp, synthetic construct
  source 1..1437: mol_type = other DNA
atgagcttactgattcgcggcggcaccattgtgacccatgatgaatcttatcgcgccgatgtgtattgtgccgatggtgttattaaagcaattggtgaaaatctggatattccggccggcgcagaagtgttagatgcctcaggccagtatcttatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccccgggcattgaagatcgtatggccgtgctgtgggatgaaggcgttaatagcggtcgtctgtcaatgcaggattttgttgccctgacctcaaccaatacggccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 146 - protein, 479 aa, synthetic construct
MSLLIRGGTIVTHDESYRADVYCADGVIKAIGENLDIPAGAEVLDASGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 147 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagattccgttttttggcaccgtttccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 148 - protein, 479 aa, synthetic construct
MSLLIRGATIVTHDESYRADVYCADGVIKAIGENLDIPAGAEVLDGSGQYLMPGGIDPHTHMQIPFFGTVSSEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 149 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtattttggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 150 - protein, 479 aa, synthetic construct
MSLLIRGATIVTHDESYRADVYCADGVIKAIGENLDIPAGAEVLDGSGQYLMPGGIDPHTHMQTPYFGTVTSEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 151 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagattccgttttatggcaccgtttccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 152 - protein, 479 aa, synthetic construct
MSLLIRGATIVTHDESYRADVYCADGVIKAIGENLDIPAGAEVLDGSGQYLMPGGIDPHTHMQIPFYGTVSSEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 153 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagattccgttttggggcaccgtttccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggagttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 154 - protein, 479 aa, synthetic construct
MSLLIRGATIVTHDESYRADVYCADGVIKAIGENLDIPAGAEVLDGSGQYLMPGGIDPHTHMQIPFWGTVSSEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 155 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagattccgttttttggcaccgttgccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 156 - protein, 479 aa, synthetic construct
MSLLIRGATIVTHDESYRADVYCADGVIKAIGENLDIPAGAEVLDGSGQYLMPGGIDPHTHMQIPFFGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 157 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagattccgttttatggcaccgttgccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 158 - protein, 479 aa, synthetic construct
MSLLIRGATIVTHDESYRADVYCADGVIKAIGENLDIPAGAEVLDGSGQYLMPGGIDPHTHMQIPFYGTVASEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 159 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagagcccgtacttcggcaccgttgcaagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 160 - protein, 479 aa, synthetic construct
MSLLIRGATIVTHDESYRADVYCADGVIKAIGENLDIPAGAEVLDGSGQYLMPGGIDPHTHMQSPYFGTVASEDFYSGTAAGLAGGTTSIIDFVIPLPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 161 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 162 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPLPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 163 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtacatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 164 - protein, 479 aa, synthetic construct
MSLLIRGATIVTHDESYRADVYCADGVIKAIGENLDIPAGAEVLDGSGQYLMPGGIDPHTHMQTPYMGTVTSEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 165 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtacatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 166 - protein, 479 aa, synthetic construct
MSLLIRGATIVTHDESYRADVYCADGVIKAIGENLDIPAGAEVLDGSGQYLMPGGIDPHTHMQTPYMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 167 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagagcccgtacttcggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccattatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 168 - protein, 479 aa, synthetic construct
MSLLIRGATIVTHDESYRADVYCADGVIKAIGENLDIPAGAEVLDGSGQYLMPGGIDPHTHMQSPYFGTVTSEDFYSGTAAGLAGGTTSIIDFVIPNPQQSLLEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 169 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 170 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 171 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtgatcgtgcagaacagcataaaccgaccgcagttaaacgt

SEQ ID NO: 172 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSDRAEQHKPTAVKR

SEQ ID NO: 173 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgcgtgatctgctgagtaaacgtgcagaacagcataaaccgaccgcagttaaacgt

SEQ ID NO: 174 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVRDLLSKRAEQHKPTAVKR

SEQ ID NO: 175 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgttgccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacatatgatggcctataaaaatgcctttatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 176 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVLPNPQQSLLEAFHQWRGWAEKSASDYGFHVAITWWSEQVREEMAELVSHHGINSFKHMMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 177 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacatcttatggcctataaaaatgcctttatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 178 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHLMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 179 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgatgccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctttatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 180 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVMPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 181 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgcttccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctttatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgaatgggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 182 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVLPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPNGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 183 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttaccagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 184 - protein, 479 aa, synthetic construct
MSLLIRGATIVTHDESYRADVYCADGVIKAIGENLDIPAGAEVLDGSGQYIMPGGIDPHTHMQTPYMGTVTSEDFYSGTAAGLAGGTTSIIDFVIPLPQQSLLEAFHQWRGWAEKSASDYGFHVAITWWSEQVREEMAELVSHHGINSFKHFMAYKNALMAADDTLVASFERCLELGAVPTVHAENGEVVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 185 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 186 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPNPQQSLLEAFHQWRGWAEKSASDYGFHVAITWWSEQVREEMAELVSHHGINSFKHFMAYKNALMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 187 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttgcaagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctacatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 188 - protein, 479 aa, synthetic construct
MSLLIRGATIVTHDESYRADVYCADGVIKAIGENLDIPAGAEVLDGSGQYIMPGGIDPHTHMQTPYMGTVASEDFYSGTAAGLAGGTTSIIDFVIPLPQQSLLEAFHQWRGWAEKSASDYGFHVAITWWSEQVREEMAELVSHHGINSFKHFMAYKNAYMAADDTLVASFERCLELGAVPTVHAENGELVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 189 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctacatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 190 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAYMAADDTLVASFERCLELGAVPTVHAENGEVVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 191 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgcagccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 192 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPQPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 193 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 194 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPLPQQSLLEAFHQWRGWAEKSASDYGFHVAITWWSEQVREEMAELVSHHGINSFKHFMAYKNALMAADDTLVASFERCLELGAVPTVHAENGEVVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 195 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctacatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 196 - protein, 479 aa, synthetic construct
MSLLIRGATIVTHDESYRADVYCADGVIKAIGENLDIPAGAEVLDGSGQYIMPGGIDPHTHMQTPYMGTVTSEDFYSGTAAGLAGGTTSIIDFVIPNPQQSLLEAFHQWRGWAEKSASDYGFHVAITWWSEQVREEMAELVSHHGINSFKHFMAYKNAYMAADDTLVASFERCLELGAVPTVHAENGEVVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 197 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 198 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPLPQQSLLEAFHQWRGWAEKSASDYGFHVAITWWSEQVREEMAELVSHHGINSFKHFMAYKNALMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 199 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 200 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 201 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctacatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 202 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPNPQQSLLEAFHQWRGWAEKSASDYGFHVAITWWSEQVREEMAELVSHHGINSFKHFMAYKNAYMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 203 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccgcccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 204 - protein, 479 aa, synthetic construct
MSLLIRGATIVTHDESYRADVYCADGVIKAIGENLDIPPGAEVLDGSGQYIMPGGIDPHTHMQTPFMGTVTSEDFYSGTAAGLAGGTTSIIDFVIPNPQQSLLEAFHQWRGWAEKSASDYGFHVAITWWSEQVREEMAELVSHHGINSFKHFMAYKNALMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 205 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttaccagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaattagtgtatcatttacagcgtaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 206 - protein, 479 aa, synthetic construct
MSLLIRGATIVTHDESYRADVYCADGVIKAIGENLDIPAGAEVLDGSGQYIMPGGIDPHTHMQTPYMGTVTSEDFYSGTAAGLAGGTTSIIDFVIPLPQQSLLEAFHQWRGWAEKSASDYGFHVAITWWSEQVREEMAELVSHHGINSFKHFMAYKNALMAADDTLVASFERCLELGAVPTVHAENGELVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 207 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 208 - protein, 479 aa, synthetic construct
MSLLIRGATIVTHDESYRADVYCADGVIKAIGENLDIPAGAEVLDGSGQYIMPGGIDPHTHMQTPYMGTVTSEDFYSGTAAGLAGGTTSIIDFVIPNPQQSLLEAFHQWRGWAEKSASDYGFHVAITWWSEQVREEMAELVSHHGINSFKHFMAYKNALMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 209 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttgcaagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 210 - protein, 479 aa, synthetic construct
MSLLIRGATIVTHDESYRADVYCADGVIKAIGENLDIPAGAEVLDGSGQYIMPGGIDPHTHMQTPYMGTVASEDFYSGTAAGLAGGTTSIIDFVIPLPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 211 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 212 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPNPQQSLLEAFHQWRGWAEKSASDYGFHVAITWWSEQVREEMAELVSHHGINSFKHFMAYKNALMAADDTLVASFERCLELGAVPTVHAENGEVVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 213 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 214 - protein, 479 aa, synthetic construct
MSLLIRGATIVTHDESYRADVYCADGVIKAIGENLDIPAGAEVLDGSGQYIMPGGIDPHTHMQTPYMGTVTSEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 215 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 216 - protein, 479 aa, synthetic construct
MSLLIRGATIVTHDESYRADVYCADGVIKAIGENLDIPAGAEVLDGSGQYIMPGGIDPHTHMQTPYMGTVTSEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 217 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctacatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 218 - protein, 479 aa, synthetic construct
MSLLIRGATIVTHDESYRADVYCADGVIKAIGENLDIPAGAEVLDGSGQYIMPGGIDPHTHMQTPYMGTVASEDFYSGTAAGLAGGTTSIIDFVIPNPQQSLLEAFHQWRGWAEKSASDYGFHVAITWWSEQVREEMAELVSHHGINSFKHFMAYKNAYMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 219 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttggaccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 220 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPNPQQSLLEAFHQWRGWTEKSASDYGFHVAITWWSEQVREEMAELVSHHGINSFKHFMAYKNALMAADDTLVASFERCLELGAVPTVHAENGEVVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 221 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccctgatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 222 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPNPQQSLLEAFHQWRGWAEKSASDYGFHVAITWWSEQVREEMAELVSHHGINSFKHFMAYKNALMAADDTLVASFERCLELGAVPTVHAENGEVVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 223 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctacatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 224 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPNPQQSLLEAFHQWRGWAEKSASDYGFHVAITWWSEQVREEMAELVSHHGINSFKHFMAYKNAYMAADDTLVASFERCLELGAVPTVHAENGEVVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 225 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 226 - protein, 479 aa, synthetic construct
MSLLIRGATIVTHDESYRADVYCADGVIKAIGENLDIPAGAEVLDGSGQYIMPGGIDPHTHMQTPYMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 227 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 228 - protein, 479 aa, synthetic construct
MSLLIRGATIVTHDESYRADVYCADGVIKAIGENLDIPAGAEVLDGSGQYIMPGGIDPHTHMQTPYMGTVTSEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 229 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtcaggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 230 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPLPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSQVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 231 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctacatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 232 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPLPQQSLLEAFHQWRGWAEKSASDYGFHVAITWWSEQVREEMAELVSHHGINSFKHFMAYKNAYMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 233 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttgcaagtgaagatttttatagtgggacagctgccggcttagcaggcgggacaacctctattattgattttgtgattccgctgccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgcctacatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 234 - protein, 479 aa, synthetic construct
MSLLIRGATIVTHDESYRADVYCADGVIKAIGENLDIPAGAEVLDGSGQYIMPGGIDPHTHMQTPYMGTVASEDFYSGTAAGLAGGTTSIIDFVIPLPQQSLLEAFHQWRGWAEKSASDYGFHVAITWWSEQVREEMAELVSHHGINSFKHFMAYKNAYMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 235 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttaccagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 236 - protein, 479 aa, synthetic construct
MSLLIRGATIVTHDESYRADVYCADGVIKAIGENLDIPAGAEVLDGSGQYIMPGGIDPHTHMQTPFMGTVTSEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 237 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 238 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 239 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtacatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 240 - protein, 479 aa, synthetic construct
MSLLIRGATIVTHDESYRADVYCADGVIKAIGENLDIPAGAEVLDGSGQYIMPGGIDPHTHMQTPYMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSAVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 241 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtcatggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 242 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSAVEGEAASRAIRIAETIGTPLYLVHVSTKEALDEITYARSHGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 243 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaacggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 244 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSAVEGEAASRAIRIAETIGTPLYLVHVSTKEALDEITYARSNGWPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 245 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaacggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 246 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSAVEGEAASRAIRIAETIGTPLYLVHVSTKEALDEITYARSNGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 247 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtcatggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 248 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSAVEGEAASRAIRIAETIGTPLYLVHVSTKEALDEITYARSHGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 249 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaacggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 250 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSAVEGEAASRAIRIAETIGTPLYLVHVSTKEALDEITYARSNGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 251 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtcatggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 252 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSAVEGEAASRAIRIAETIGTPLYLVHVSTKEALDEITYARSHGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 253 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtcatggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 254 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSPVEGEAASRAIRIAETIGTPLYLVHVSTKEALDEITYARSHGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 255 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtcatggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 256 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSAVEGEAASRAIRIAETIGTPLYLVHVSTKEALDEITYARSHGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 257 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggtccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaacggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 258 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMVQGITGPEAHPLSRPSAVEGEAASRAIRIAETIGTPLYLVHVSTKEALDEITYARSNGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 259 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtaacggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 260 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSPVEGEAASRAIRIAETIGTPLYLVHVSTKEALDEITYARSNGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 261 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtaacggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 262 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSPVEGEAASRAIRIAETIGTPLYLVHVSTKEALDEITYARSNGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 263 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtaacggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 264 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSAVEGEAASRAIRIAETIGTPLYLVHVSTKEALDEITYARSNGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 265 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtcatggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccccgggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 266 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSPVEGEAASRAIRIAETIGTPLYLVHVSTKEALDEITYARSHGWPVYGEVLAGHLLLDDSVYQHPDWQTAAGYVMSPPFRPRGHQDALWHGLQSGNLHTTATDHCCFCAEQKAAGRDDFSKIPPGTPGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 267 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtcatggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 268 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSPVEGEAASRAIRIAETIGTPLYLVHVSTKEALDEITYARSHGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 269 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtcatggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 270 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSAVEGEAASRAIRIAETIGTPLYLVHVSTKEALDEITYARSHGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 271 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtcatggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 272 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSPVEGEAASRAIRIAETIGTPLYLVHVSTKEALDEITYARSHGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 273 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 274 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSPVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 275 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaagtggtgtatcatttacagcgcaaactgatggcccagggtattaccggcccggaagcacatccgctgagtcgtccgagtgcggtggaaggcgaagcagcctctcgcgcaattcgtattgccgaaaccattggcaccccgttatatctggttcatgtgtcaaccaaagaagccttagatgaaattacctatgcacgtagtcatggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 276 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEVVYHLQRKLMAQGITGPEAHPLSRPSAVEGEAASRAIRIAETIGTPLYLVHVSTKEALDEITYARSHGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 277 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtaacggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 278 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSPVEGEAASRAIRIAETIGTPLYLVHVSTKEALDEITYARSNGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 279 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtaaaggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 280 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSPVEGEAASRAIRIAETIGTPLYLVHVSTKEALDEITYARSKGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 281 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatggtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtcatggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 282 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEMVYHLQRKLMAQGITGPEAHPLSRPSPVEGEAASRAIRIAETIGTPLYLVHVSTKEALDEITYARSHGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 283 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatttaatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaattagtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtcatggctggccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 284 - protein, 479 aa, synthetic construct
MSLLIRGATIVTHDESYRADVYCADGVIKAIGENLDIPAGAEVLDGSGQYLMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGELVYHLQRKLMAQGITGPEAHPLSRPSPVEGEAASRAIRIAETIGTPLYLVHVSTKEALDEITYARSHGWPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

SEQ ID NO: 285 - DNA, 1437 bp, synthetic construct
  source 1..1437: mol_type = other DNA
atgagcttactgattcgcggcgccaccattgtgacccatgatgaatcttatcgcgccgatgtgtattgtgccgatggtgttattaaagcaattggtgaaaatctggatattccggccggcgcagaagtgttagatggctcaggccagtatatcatgccgggcggcattgatccgcatacccacatgcagacgccgtttatgggcaccgttgcaagtgaagatttttatagtgggacagctgccggcttagcaggcgggacaacctctattattgattttgtgattccgaatccgcagcagtctttactggaagcctttcatcagtggcgcggttgggccgaaaaatctgcctcagattatggctttcatgttgcaattacctggtggagcgaacaggttcgcgaagaaatggcagaactggtttcacatcatggtattaatagctttaaacattttatggcctataaaaatgccttcatggcagccgatgataccttagttgcgagttttgaacgctgtctggaactgggtgcagttccgaccgttcatgccgaaaatggtgaaatcgtgtatcatttacagcgcaaactgatggcccagggtattaccggcccggaagcacatccgctgagtcgtccgagtccggtggaaggcgaagcagcctctcgcgcaattcgtattgccgaaaccattggcaccccgttatatctggttcatgtgtcaaccaaagaagccttagatgaaattacctatgcacgtagtcatggccagccggtgtatggcgaagttctggccggtcatctgctgctggatgatagcgtgtatcagcatccggattggcagaccgcagcaggctatgttatgtctccgccgtttcgtccgcgtggtcatcaggatgccctgtggcatggcttacagagcggtaatttacataccaccgccaccgatcattgttgtttttgtgcagaacagaaagccgcaggtcgtgatgatttttctaaaattccgccggggaccgccggcattgaagatcgtatggccgtgctgtgggatgaaggcgttaatagcggtcgtctgtcaatgcaggattttgttgccctgacctcaaccaataccgccaaaatttttaatctgtatccgcgtaaaggcgcaattcgcgtgggtgcagatgccgatttagtgctgtgggacccgcagggcacccgtaccattagcgccaaaacccatcatcagcaggttgattttaatatttttgaaggtaaaaccgtgactggcgttccgtcacataccgtttctcagggtcgcgttgtttgggccgatggcgatctgcgtgcagaacgcggtgccggtcgctatattgaacgcccggcctatccggcagtgtttgatctgctgagtaaacgtgcagaacagcataaaccgaccgcagttaaacgt

SEQ ID NO: 286 - protein, 479 aa, synthetic construct
MSLLIRGATIVTHDESYRADVYCADGVIKAIGENLDIPAGAEVLDGSGQYIMPGGIDPHTHMQTPFMGTVASEDFYSGTAAGLAGGTTSIIDFVIPNPQQSLLEAFHQWRGWAEKSASDYGFHVAITWWSEQVREEMAELVSHHGINSFKHFMAYKNAFMAADDTLVASFERCLELGAVPTVHAENGEIVYHLQRKLMAQGITGPEAHPLSRPSPVEGEAASRAIRIAETIGTPLYLVHVSTKEALDEITYARSHGQPVYGEVLAGHLLLDDSVYQHPDWQTAAGYVMSPPFRPRGHQDALWHGLQSGNLHTTATDHCCFCAEQKAAGRDDFSKIPPGTAGIEDRMAVLWDEGVNSGRLSMQDFVALTSTNTAKIFNLYPRKGAIRVGADADLVLWDPQGTRTISAKTHHQQVDFNIFEGKTVTGVPSHTVSQGRVVWADGDLRAERGAGRYIERPAYPAVFDLLSKRAEQHKPTAVKR

(19) United States
(12) Patent Application Publication
(10) Pub. No.: US 2025/0043323 A1
(43) Pub. Date: Feb. 6, 2025

(54) BIOCATALYSTS AND METHODS FOR THE SYNTHESIS OF PREGABALIN INTERMEDIATES

(71) Applicant: Enzymaster (Ningbo) Bio-Engineering Co., Ltd, Ningbo (CN)

(72) Inventors: Yingxin ZHANG, Ningbo (CN); Haibin CHEN, Ningbo (CN); Marco BOCOLA, Ningbo (CN); Baoqin CAI, Ningbo (CN); Zhaoqi ZHANG, Ningbo (CN); Xiao LUO, Ningbo (CN); Yaoyao JI, Ningbo (CN); Chengxiao ZHANG, Ningbo (CN); Ruimei HONG, Ningbo (CN)

(73) Assignee: Enzymaster (Ningbo) Bio-Engineering Co., Ltd, Ningbo (CN)

(21) Appl. No.: 18/711,814

(22) PCT Filed: Oct. 30, 2022
(86) PCT No.: PCT/CN2022/128468

§ 371 Date: May 20, 2024

(30) Foreign Application Priority Data
Nov. 21, 2021 (CN) 202111381310.9

(51) Int. Cl. C12P 17/10; C12N 9/86; C12P 13/00

(52) U.S. Cl. CPC C12P 17/10; C12N 9/86; C12P 13/001

(57) ABSTRACT

Provided is an engineered polypeptide capable of catalyzing the asymmetric hydrolysis of 3-isobutylglutarimide to generate (R)-(−)-3-(carbamoylmethyl)-5-methylhexanoic acid. The engineered polypeptide has high stereoselectivity, high catalytic activity, good process stability and thermal stability, and tolerance to high product concentrations, and has good prospects for industrial applications.

### PRIORITY

This application corresponds to the U.S. National phase of International Application No. PCT/CN2022/128468, filed Oct. 30, 2022, which, in turn, claims priority to Chinese Patent Application No. 2021 11381310.9 filed Nov. 21, 2021, the contents of which are incorporated by reference herein in their entirety.

### SEQUENCE LISTING

The instant application contains a Sequence Listing that has been submitted in XML format via EFS-Web and is hereby incorporated by reference in its entirety. Said XML copy, created on May 20, 2024, is named LNK_255US_SEQLIST.XML and is 514,856 bytes in size.

### TECHNICAL FIELD

The present invention relates to a biocatalyst and a method for preparing a pregabalin intermediate by using the biocatalyst.

### BACKGROUND OF THE PRESENT INVENTION

Pregabalin is a chiral small-molecule drug compound with the chemical name S-(+)-3-isobutyl gamma-aminobutyric acid. It is associated with endogenous inhibitory neurotransmitters and has antiepileptic activity, and it is therefore commonly used for the treatment of antiepilepsy and neuralgia. Pregabalin was originally manufactured by Pfizer in the United States, and it was approved by the European Union for the treatment of partial seizures in July 2004, and approved by the U.S. FDA in 2005. Its original synthetic route is shown in FIG. 1.

One of the most important indicators for the production of pregabalin API is chiral purity. The synthesis methods of pregabalin API and its intermediates in existing patents and literature are mainly divided into three categories: chemical/enzymatic resolution route, asymmetric synthesis route and chiral source synthesis route, of which the former two routes are used more frequently. In the resolution route, the ee value of the product is relatively low and the other enantiomer needs to be racemized for reuse, resulting in a low yield of the final qualified product. For example, CN102102114B discloses a technology for the preparation of pregabalin intermediates by lipase resolution, the conversion of the resolution step is about 40-45%, and the overall yield is only about 30%. This route is shown in FIG. 2.

In contrast, asymmetric synthesis methods that directly introduce chirality into the products have higher raw material utilization and can be accomplished with chiral catalysts or enzymes. However, chemical asymmetric synthesis methods require the use of expensive chiral catalysts and the process is often complicated and cumbersome, such as the original route developed by Pfizer, which requires nine steps. The synthesis process disclosed in patent CN105753726B has only 4 steps, but it requires the use of chiral thiourea ammonium salt as a catalyst, involving harsh processes such as hydrogenation. This route is shown in FIG. 3.

Therefore, there is an urgent need to develop a more sustainable and greener method to produce pregabalin. CN111944856A discloses a novel route for the synthesis of pregabalin intermediates, i.e., 3-isobutylpiperidine-2,6-dione is asymmetrically hydrolyzed by hydantoinase to obtain (R)-(−)-3-(carbamoylmethyl)-5-methylhexanoic acid (R-CMH) with high chiral purity (as shown in FIG. 4). This enzymatic reaction can produce R-CMH with ee ≥99%, which avoids the resolution or racemization steps, shortens the overall synthetic path, improves the utilization of raw materials, is environmentally friendly, and effectively reduces overall costs. However, the catalytic performance of hydantoinase disclosed in CN111944856A is not satisfactory, where the enzyme loading in the reaction is high, and the space-time yield of the hydantoinase reaction is low.

To overcome these deficiencies, the present invention discloses a series of engineered hydantoinase polypeptides developed by directed evolution technology, which greatly reduces the enzyme loading in the hydantoinase reaction, enables simple and efficient enzymatic reaction process and workup process, and greatly improves the space-time yield.

### SUMMARY OF THE PRESENT INVENTION

The present invention provides engineered polypeptides with high stereoselectivity, high catalytic activity, good process stability & thermal stability as well as tolerance to high product concentrations, which can be used to catalyze the asymmetric hydrolysis of 3-isobutylglutarimide (structure shown as compound A1 in FIG. 5) to generate (R)-(−)-3-(carbamoylmethyl)-5-methylhexanoic acid (structure shown as compound A2 in FIG. 5). Also provided are the genes for the engineered polypeptides, a recombinant expression vector containing the genes, an engineered strain and an efficient method for the preparation of the engineered polypeptides, and a reaction process for the preparation of A2 using the engineered polypeptides.

Through experimental studies, the inventors identified a wild-type hydantoinase (GenBank: KF268426.1) from Pseudomonas fluorescens with the amino acid sequence shown in SEQ ID NO: 2. Compared to the hydantoinase disclosed in CN111944856A, SEQ ID NO: 2 shows better activity of catalyzing the asymmetric hydrolysis of A1 to produce A2. Although SEQ ID NO: 2 is an enzyme with superior activity for the reaction shown in FIG. 5 among many wild-type hydantoinases studied by the inventors, it is still far from industrial application and its performance in various aspects needs to be improved. The study of this wild-type hydantoinase was reported in Appl Biochem Biotechnol (2016) 179:1-15, which showed that the optimal pH of this wild-type hydantoinase is between 8.5 and 9.5 when it catalyzes the hydrolysis of substituted hydantoins; at pH<7.5, the activity decreased significantly. Its thermal stability was also poor; its half-lives at 50° C., 55° C. and 60° C. were 2.23 h, 1.44 h and 0.78 h, respectively, which were not favorable for the production and storage of enzyme in large quantity. The inventors found that in the absence of any catalyst, 3-isobutylglutarimide spontaneously hydrolyzes to produce racemic 3-(carbamoylmethyl)-5-methylhexanoic acid. The rate of this spontaneous hydrolysis is strongly dependent on pH, and it is significant at pH >8.5. The resulting racemic product contains the undesired isomer (S)-(−)-3-(carbamoylmethyl)-5-methylhexanoic acid, which affects the chiral purity (i.e., ee value) of the final product, so the spontaneous hydrolysis of 3-isobutylglutarimide is strongly to be avoided by the present invention. The spontaneous hydrolysis of 3-isobutylglutarimide is almost undetectable at pH 7.0, so the reaction shown in FIG. 5 needs to be carried out at pH 7.0.

In addition to the need to improve the activity, thermal stability and process stability (at pH 7.0) of SEQ ID NO: 2 for catalyzing the reaction shown in FIG. 5, the inventors found that the activity of SEQ ID NO: 2 was severely inhibited when the product concentration in the reaction system accumulated to a certain level, which limits the further improvement of the space-time yield of A2. So, overcoming the product inhibition of SEQ ID NO: 2 (or in other words, improving the tolerance of SEQ ID NO:2 to high product concentrations) is also to be addressed. Using directed evolution technology with computer-aided design and screening, the inventors have engineered SEQ ID NO:2 and obtained a series of engineered polypeptides with high stereoselectivity, high catalytic activity, good thermal stability & process pH stability, as well as good tolerance to high product concentrations. These engineered polypeptides include amino acid sequences having one or more residue differences compared to the reference sequence of SEQ ID NO:2, these residue differences occur at amino acid positions that affect multiple different functional properties of the enzyme, including catalytic activity, stereoselectivity, substrate and/or product tolerance, thermal stability, reaction process stability (including pH fluctuation, ionic strength, solvent tolerance, etc.), recombinant expression effects, etc. and other properties that affect the preparation and catalytic performance of the enzyme, as well as various combinations of these properties.

In some embodiments, the engineered polypeptide may comprise an amino acid sequence having at least 90% sequence identity to the polypeptide of SEQ ID NO: 2 and differing from SEQ ID NO: 2 in one or more residues at residue positions selected from: X8, X39, X46, X51, X62, X63, X64, X66, X67, X71, X73, X95 X97, X113, X152, X159, X189, X199, X201, X215, X254, X255, X257, X263, X264, X265, X266, X267, X288, X292, X320, X329, X336, X337, X340, X462, X467, X474 X476, X479. In some embodiments, the amino acid residue differences compared to SEQ ID NO: 2 are selected from the group consisting of: A8G, A39P, G46A, L51V, L51I, M62L, Q63E, L64I, L64T, L64S, L64A, F66Y, F66L, M67W, M67Y, M67F, A71T, A71S, E73D, 195V, 195L, 195M, N97G, N97D, N97L, N97Q, A113T, F152Y, F152M, F152L, I159L, I159F, I159Y, L189I, L189V, L189, Q, A199, Q215P, S254Q, S254L, S254N, S254G, S254F, K255F, K255Y, K255H, K255N, Q257W, V263T, L264C, A265P, G266Q, H267Y, M288C, F292L, F320S, F320L, R329A, R329L, R329Y, P336M, P336L, P336Q, N337P, A340P, F462R, K467D, P474W, A476P, R479Q, R479L, R479P; or also on the basis of these differences, containing 1, 2, 3, 4, 5, 6, 7, 8, 9, 10, 11, 12, 13, 14, 15 16, 18, 20, 21, 22, 23, 24, 25 or more insertions or deletions of amino acid residues.

As provided herein, in some embodiments, the disclosed amino acid differences may be used alone or in various combinations to produce engineered polypeptides with improved enzymatic properties. In some embodiments, the engineered polypeptide comprises an amino acid sequence having at least 90% sequence identity to the reference sequence SEQ ID NO: 2 and at least one residue difference at residue position X64 as compared to SEQ ID NO: 2. In some embodiments, the amino acid residue at residue position X64 is selected from the group consisting of I, T, S, and A.

More specifically, in some embodiments, the engineered polypeptides improved on the basis of SEQ ID NO: 2 comprise polypeptides consisting of the amino acid sequences corresponding to SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262 264, 266, 268, 270, 272, 274, 276, 278, 280, 282, 284, 286.

In some embodiments, the improved engineered polypeptide comprises amino acid sequences that have at least 90%, 91%, 92%, 93%, 94%, 95%, 96%, 97%, 98%, 99% or more sequence identity of the reference sequence of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270 272, 274, 276, 278, 280, 282, 284, 286.

The identity between two amino acid sequences or two nucleotide sequences can be obtained by algorithms commonly used in the art, either by using the NCBI Blastp and Blastn software based on default parameters or by using the Clustal W algorithm (Nucleic Acid Research, 22 (22): 4673-4680, 1994). For example, using the Clustal W algorithm, the amino acid sequence identity of SEQ ID NO:2 and SEQ ID NO:184 is 97.9%.

In another aspect, the present invention provides polynucleotide sequences encoding engineered polypeptides. In some embodiments, the polynucleotide may be a portion of an expression vector having one or more control sequences for expression of the engineered polypeptide. In some embodiments, the polynucleotide may comprise a polynucleotide sequence corresponding to the sequences shown in SEQ ID No: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273 275, 277, 279, 281, 283, 285.

As known to those of skill in the art, due to the degeneracy of nucleotide codons, the polynucleotide sequences encoding the amino acid sequence of SEQ ID No: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286 are not limited to SEQ ID No: 3, 5, 7, 9, 11, 13, 15, 17, 19, 21, 23, 25, 27, 29, 31, 33, 35, 37, 39, 41, 43, 45, 47, 49 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255 257, 259, 261, 263, 265, 267, 269, 271, 273, 275, 277, 279, 281, 283, 285. The nucleic acid sequence of the hydantoinase gene of the present invention can also be any other nucleic acid sequence encoding the amino acid sequence shown in SEQ ID No: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240 242, 244, 246, 248, 250, 252, 254, 256, 258, 260, 262, 264, 266, 268, 270, 272, 274, 276, 278, 280, 282, 284, 286.

In another aspect, the present disclosure provides expression vectors and host cells comprising a polynucleotide encoding an engineered polypeptide or capable of expressing an engineered polypeptide. In some embodiments, the host cell may be a bacterial host cell, such as E. coli. The host cell can be used to express and isolate the engineered polypeptide as described herein, or alternatively, to react directly to convert substrates into products.

In some embodiments, the engineered polypeptide in the form of whole cells, crude extracts, isolated polypeptides, or purified polypeptides may be used alone, or in immobilized form (e.g., immobilized on a resin).

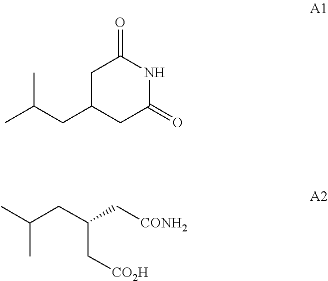

The present disclosure also provides methods for converting a compound shown in structural formula A1 to a chiral compound shown in structural formula A2 using an engineered polypeptide disclosed herein, the chiral compound shown in structural formula A2 being in an enantiomeric excess over the other isomers, said methods comprising contacting the compound of structural formula A1 with an engineered polypeptide under reaction conditions suitable for converting A1 to A2, wherein said engineered polypeptide is engineered polypeptide as described herein. In some embodiments, said engineered polypeptide has at least 90%, 91%, 92%, 93%, 94%, 95%, 96%, 97%, 98%, 99% or more sequence identity to SEQ ID NO: 2 and is capable of converting the compound of structural formula A1 to the compound of structural formula A2.

In some embodiments, the compound of structural formula A2 is produced in an enantiomeric excess of at least 97%, 98%, or 99% or more.

Specific embodiments of engineered polypeptides for use in this method are provided further in the detailed description. The engineered polypeptide applicable in the above methods may comprise an amino acid sequence selected from those having at least 90% sequence identity to SEQ ID NO: 2 and having one or more residue differences compared to SEQ ID NO: 2 at residue positions selected from: X8, X39, X46, X51, X62, X63, X64, X66, X67, X71, X73, X95 X97, X113, X152, X159, X189, X199, X201, X215, X254, X255, X257, X263, X264, X265, X266, X267, X288, X292, X320, X329, X336, X337, X340, X462, X467, X474 X476, X479. In some embodiments, the amino acid residue differences compared to SEQ ID NO: 2 are selected from the group consisting of: A8G, A39P, G46A, L51V, L51I, M62L, Q63E, L64I, L64T, L64S, L64A, F66Y, F66L, M67W, M67Y, M67F, A71T, A71S, E73D, 195V, 195L, 195M, N97G, N97D, N97L, N97Q, A113T, F152Y, F152M, F152L, I159L, I159F, I159Y, L189I, L189V, L189, Q, A199, Q215P, S254Q, S254L, S254N, S254G, S254F, K255F, K255Y, K255H, K255N, Q257W, V263T, L264C, A265P, G266Q, H267Y, M288C, F292L, F320S, F320L, R329A, R329L, R329Y, P336M, P336L, P336Q, N337P, A340P, F462R, K467D, P474W, A476P, R479Q, R479L, R479P; or also on the basis of these differences, containing 1, 2, 3, 4, 5, 6, 7, 8, 9, 10, 11, 12, 13, 14, 15 16, 18, 20, 21, 22, 23, 24, 25 or more insertions or deletions of amino acid residues.

In some embodiments, the engineered polypeptide applicable in the above methods may comprise amino acid sequences selected from the group corresponding to SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 66, 70, 72, 74 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286.

Any of the methods of using an engineered polypeptide for producing the compound of formula A2 as disclosed herein may be performed under a range of suitable reaction conditions, said range of suitable reaction conditions including, but not limited to, pH, temperature, buffer, solvent system, substrate loading, polypeptide loading, pressure, and reaction time. For example, in some embodiments, preparation of the compound of formula A2 may be performed wherein suitable reaction conditions include (a) a substrate loading of about 1 g/L to 400 g/L of compound A1; (b) a loading of about 0.1 g/L to 50 g/L of the engineered polypeptide; (d) a pH of about 6.0 to about 8.5; and (d) a temperature of about 10° C. to about 60° C.

In some embodiments, the engineered polypeptide is capable of converting compound A1 to compound A2 under appropriate reaction conditions, having at least about 2-fold, 3-fold, 4-fold, 5-fold, 10-fold, 15-fold, 20-fold, or more-fold increased activity relative to the reference polypeptide of SEQ ID NO:2. In some embodiments, the engineered polypeptide is capable of converting compound A1 to compound A2 under appropriate reaction conditions in a reaction time of about 48 hours, about 36 hours, about 24 hours, or less, with at least about 5 g/L h⁻¹, 10 g/L h⁻¹, 15 g/L h⁻¹, 20 g/L h⁻¹ or higher space-time yield.

### BRIEF DESCRIPTION OF THE FIGURES

FIG. 1 depicts the original synthetic route for pregabalin.

FIG. 2 depicts the synthesis of pregabalin and its intermediates by lipase resolution route.

FIG. 3 depicts the chemical asymmetric synthesis of pregabalin and its intermediates.

FIG. 4 depicts the asymmetric synthesis of pregabalin intermediate by hydantoinase and subsequent Hofmann reaction to produce pregabalin API.

FIG. 5 depicts the hydantoinase provided in present invention catalyze the asymmetric hydrolysis of 3-isobutylglutarimide to generate (R)-(−)-3-(carbamoylmethyl)-5-methylhexanoic acid.

FIG. 6 depicts the results of protein electrophoresis.

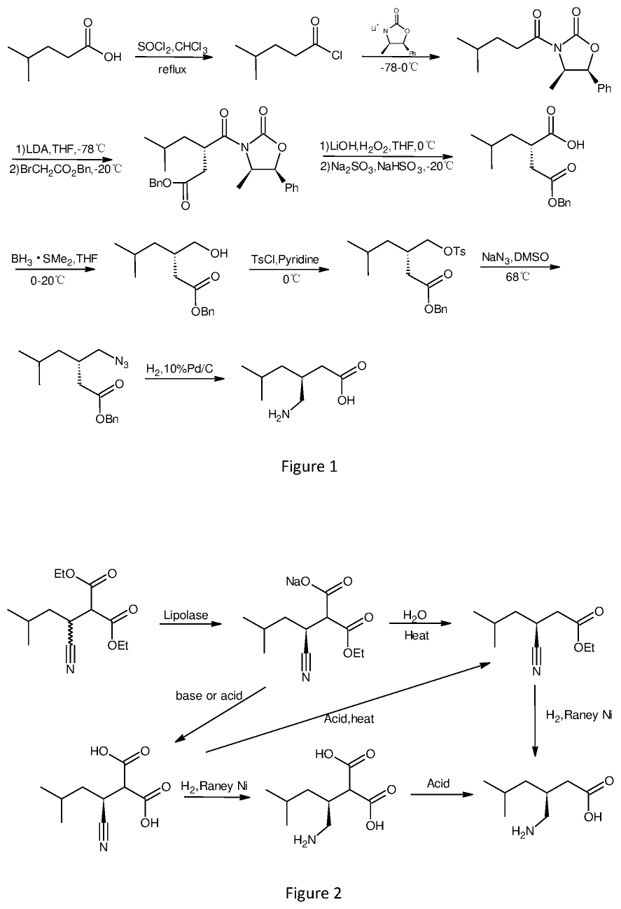

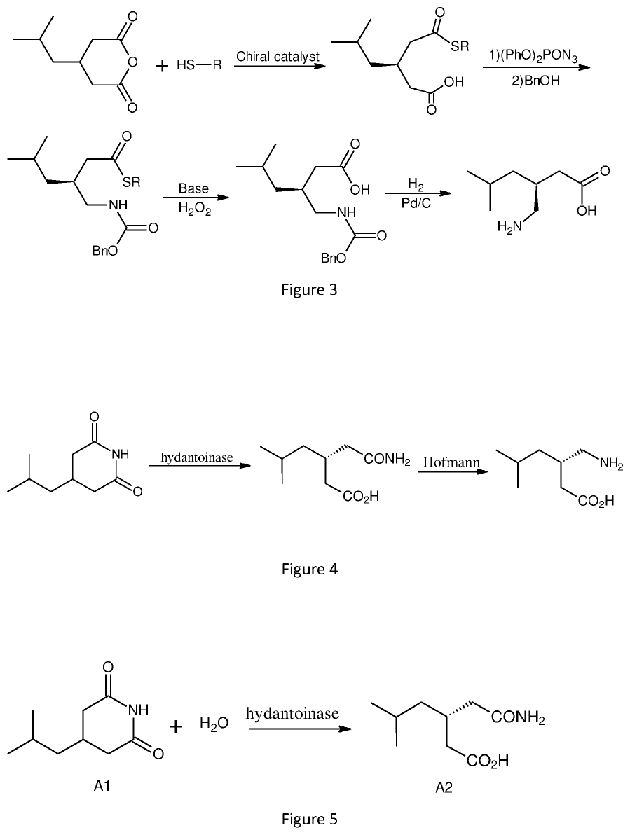

### DETAILED DESCRIPTION OF THE PREFERRED EMBODIMENTS

### 1. Definitions

With respect to this disclosure, unless otherwise expressly defined, technical terms and scientific terms used in the specification herein have the meanings commonly understood by those of ordinary skill in the art.

The terms “protein,” “polypeptide,” and “peptide” are used interchangeably herein to refer to a polymer of at least two amino acids covalently linked by an amide bond, regardless of length or post-translational modifications (e.g., glycosylation, phosphorylation, lipidation, myristoylation, ubiquitination, etc.). The definition includes D-amino acids and L-amino acids, and mixtures of D-amino acids and L-amino acids.

The terms “engineered hydantoinase,” “engineered hydantoinase polypeptide,” “improved hydantoinase polypeptide,” and “engineered polypeptide” are used interchangeably herein. “Polynucleotide” and “nucleic acid” are used interchangeably herein.

The term “coding sequence” refers to the nucleic acid portion (e.g., a gene) that encodes an amino acid sequence of a protein.

“Naturally occurring” or “wild-type” refers to the form found in nature. For example, a naturally occurring or wild-type polypeptide or polynucleotide sequence is a sequence that exists in an organism that is isolable from a natural source and has not been intentionally modified by artificial manipulation.

“Recombinant” or “engineered” or “non-naturally occurring”, when used in reference to, for example, a cell, nucleic acid or polypeptide, refers to a material that is, or corresponds to, the natural or inherent form of the material, that has been altered in a manner not found in nature, or is identical to it but is produced or obtained from synthetic material and/or by manipulation using recombinant technology.

The terms “sequence identity” and “homology” are used interchangeably herein to refer to comparisons between polynucleotides or polypeptides (“sequence identity” and “homology” are typically expressed as a percentage) and is determined by comparing two optimally aligned sequences on a comparison window, where the portion of the polynucleotide or polypeptide sequence in the comparison window may include additions or deletions (i.e., gaps) compared to the reference sequence for optimal alignment of the two sequences. The percentage may be calculated by determining the number of positions where identical nucleic acid bases or amino acid residues occur in the two sequences to produce the number of matching positions, dividing the number of matching positions by the total number of positions in the comparison window and multiplying the result by 100 to obtain the sequence identity percentage. Optionally, the percentage may be calculated by determining the number of positions where the same nucleic acid base or amino acid residue is present in both sequences or the number of positions where the nucleic acid base or amino acid residue is aligned with gaps to obtain the number of matching positions, dividing that number of matching positions by the total number of positions in the comparison window, and multiplying the result by 100 to obtain the percentage of sequence identity. Those skilled in the art will recognize that many established algorithms exist that can be used to align two sequences. The optimal alignment of sequences for comparison can be done, for example, by the local homology algorithm of Smith and Waterman, 1981, Adv. Appl. Math. 2:482, by the homology comparison algorithm of Needleman and Wunsch, 1970, J. Mol. Biol. 48:443, by the homology comparison algorithm of Pearson and Lipman, 1988, Proc. Natl. Acad. Sci. USA85:2444, by computerized implementations of these algorithms (GAP, BESTFIT, FASTA, or TFASTA in the GCG Wisconsin package) or by visual inspection (see, generally, Current Protocols in Molecular Biology, edited by F. M. Ausubel et al, Current Protocols, a joint venture between Greene Publishing Associates Inc. and John Wiley & Sons, Inc. (1995 supplement) (Ausubel)). Examples of algorithms suitable for determining sequence identity and percent sequence similarity are the BLAST and BLAST2.0 algorithms, which are described in Altschul et al, 1990, J. Mol. Biol. 215:403-410 and Altschul et al, 1977, Nucleic Acids Res. 3389-3402, respectively. The software used to perform the BLAST analysis is publicly available through the National Center for Biotechnology Information (NCBI) website. The algorithm involves first identifying high scoring sequence pairs (HSPs) by identifying short words of length W in the query sequence, which either match or satisfy some positive-valued threshold scores T when aligned with a word of the same length in the database sequence. T is referred to as, the neighborhood word score threshold (Altschul et al., Supra). These initial neighborhood word hits serve as seeds for initiating searches to find longer HSPs that contain them. The word hits are then extended in both directions along each sequence for as far as the cumulative alignment score can be increased. For nucleotide sequences, the cumulative scores are calculated using the parameters M (reward score for matched pair of residues; always>0) and N (penalty score for mismatched residues; always <0). For amino acid sequences, a scoring matrix is used to calculate the cumulative score. The extension of the word hits in each direction are halted when: the cumulative alignment score falls off by the quality X from its maximum achieved value; the cumulative score goes 0 or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, the expected value (E) of 10, M=5, N=−4, and a comparison of both strands as a default value. For amino acid sequences, the BLASTP program uses as defaults the wordlength (W) of 3, the expected value (E) of 10 and the BLOSUM62 scoring matrix (see Henikoff and Henikoff, 1989, Proc Natl Acad Sci USA 89: 10915). Exemplary determination of sequence alignments and % sequence identity can employ the BESTFIT or GAP programs in the GCG Wisconsin Software package (Accelrys, Madison WI), using the default parameters provided.

“Reference sequence” refers to a defined sequence that is used as a basis for sequence comparison. The reference sequence may be a subset of a larger sequence, for example, a full-length gene or a fragment of a polypeptide sequence. In general, a reference sequence is at least 20 nucleotides or amino acid residues in length, at least 25 residues long, at least 50 residues in length, or the full length of the nucleic acid or polypeptide. Because two polynucleotides or polypeptides may each (1) comprise a sequence (i.e., a portion of the complete sequence) that is similar between two sequences, and (2) may further comprise sequences that is divergent between the two sequences, sequence comparisons between two (or more) polynucleotides or polypeptides are typically performed by comparing the sequences of the two polynucleotides or polypeptides over a “comparison window” to identify and compare local regions of sequence similarity. In some embodiments, a “reference sequence” is not intended to be limited to a wild-type sequence, and may comprise engineered or altered sequences. For example, “a reference sequence having a threonine at a residue corresponding to X64 based on SEQ ID NO:2” refers to a reference sequence wherein the corresponding residue (being a leucine) at X64 in SEQ ID NO:2 has been altered to a threonine.

“Comparison window” refers to a conceptual segment of at least about 20 contiguous nucleotide positions or amino acid residues, wherein the sequence may be compared to a reference sequence of at least 20 contiguous nucleotides or amino acids and wherein the portions of the sequence in the comparison window may comprise 20% or less additions or deletions (i.e., gaps) compared to the reference sequence (which does not comprise additions or deletions) for optimal alignment of the two sequences. The comparison window can be longer than 20 contiguous residues, and optionally include 30, 40, 50, 100 or more residues.

In the context of the numbering for a given amino acid or polynucleotide sequence, “corresponding to,” “reference to” or “relative to” refers to the numbering of the residues of a specified reference when the given amino acid or polynucleotide sequence is compared to the reference sequence. In other words, the residue number or residue position of a given sequence is designated with respect to the reference sequence, rather than by the actual numerical position of the residue within the given amino acid or polynucleotide sequence. For example, a given amino acid sequence such as the amino acid sequence of an engineered hydantoinase can be aligned to a reference sequence, by introducing gaps to optimize the residue match between the two sequences. In these cases, the numbering of the residue in a given amino acid or polynucleotide sequence is made with respect to the reference sequence to which it has been aligned, despite the presence of a gap position.

An “amino acid difference” or “residue difference” refers to a difference in an amino acid residue at a position of a polypeptide sequence relative to an amino acid residue at a corresponding position in a reference sequence. The position of an amino acid difference is generally referred to herein as “Xn”, where n refers to the corresponding position in the reference sequence on which the residue difference is based. For example, “residue difference at position X64 compared to SEQ ID NO: 2” refers to the difference in amino acid residues at the polypeptide position corresponding to position 64 of SEQ ID NO:2. Thus, if the reference polypeptide of SEQ ID NO:2 has a leucine at position 64, then “residue difference at position X64 compared to SEQ ID NO:2” refers to an amino acid substitution of any residue other than a leucine at the position of the polypeptide corresponding to position 64 of SEQ ID NO:2. In most of the examples herein, the specific amino acid residue difference at the position is indicated as “XnY”, wherein “Xn” refers to the corresponding position as described above, and “Y” is the single letter identifier of the amino acid found in the engineered polypeptide (i.e., a different residue than in the reference polypeptide). In some examples (e.g., in Table 1), the present disclosure also provides specific amino acid differences indicated by the conventional symbol “AnB”, where A is a single letter identifier of a residue in the reference sequence, “n” is the number of residue position in the reference sequence, and B is the single letter identifier for the residue substitution in the sequence of the engineered polypeptide. In some examples, the polypeptide of the present disclosure may comprise one or more amino acid residue differences relative to a reference sequence, which is indicated by a list of specific positions at which residue differences are present exist relative to the reference sequence.

“Deletion” refers to the modification of a polypeptide by removing one or more amino acids from a reference polypeptide. Deletions can include the removal of one or more amino acids, two or more amino acids, five or more amino acids, ten or more amino acids, fifteen or more amino acids, or twenty or more amino acids, up to 10% of the total number of amino acids of the enzyme, or up to 20% of the total number of amino acids making up the reference enzyme while retaining the enzymatic activity of the engineered hydantoinase and/or retaining the improved properties of the engineered hydantoinase. Deletion may involve the internal portion and/or the terminal portion of the polypeptide. In various embodiments, deletions may include a contiguous segment or may be discontinuous.

“Insertion” refers to a modification of the polypeptide by adding one or more amino acids from the reference polypeptide. In some embodiments, the improved engineered hydantoinase comprises insertions of one or more amino acids to into a naturally occurring hydantoinase polypeptide, as well as insertions of one or more amino acids to other engineered hydantoinase polypeptides. The insertion may be made in the internal portion of the polypeptide, or into the carboxyl or amino terminus. As used herein, insertions include fusion proteins known in the art. The insertion may be a contiguous segment of amino acids or be separated by one or more amino acids in naturally-occurring or engineered polypeptides.

As used herein, “fragment” as used herein refers to a polypeptide having an amino terminal and/or carboxyl terminal deletion, but where the remaining amino acid sequence is identical to the corresponding position in the sequence. Fragments may be at least 10 amino acids long, at least 20 amino acids long, at least 50 amino acids long or longer, and up to 70%, 80%, 90%, 95%, 98% and 99% of the full-length hydantoinase polypeptide.

An “isolated polypeptide” refers to a polypeptide that is substantially separated from other substances with which it is naturally associated, such as proteins, lipids, and polynucleotides. The term comprises polypeptides that have been removed or purified from their naturally occurring environment or expression system (e.g., in host cells or in vitro synthesis). Engineered hydantoinase polypeptides may be present in the cell, in the cell culture medium, or prepared in various forms, such as lysates or isolated preparations. As such, in some embodiments, the engineered hydantoinase polypeptide may be an isolated polypeptide.

“Chiral center” refers to a carbon atom connecting four different groups.

“Stereoselectivity” refers to the preferential formation of one stereoisomer over the other in a chemical or enzymatic reaction. Stereoselectivity can be partial, with the formation of one stereoisomer is favored over the other; or it may be complete where only one stereoisomer is formed. When the stereoisomers are enantiomers, the stereoselectivity is referred to as enantioselectivity. It is often reported as “enantiomeric excess” (ee for short). When the stereoisomers are diastereomers, the stereoselectivity is referred to as diastereoselectivity. It is often reported as “diastereomeric excess” (de for short). The fraction, typically a percentage, is generally reported in the art as optionally reported as the enantiomeric excess (i.e., ee) derived therefrom according to the following formula: {major enantiomer concentration−minor enantiomer concentration}/{major enantiomer concentration+minor enantiomer concentration}.

The terms “stereoisomers”, “stereoisomeric forms” and similar expressions are used interchangeably herein to refer to all isomers resulting from a difference in orientation of atoms in their space only. These include enantiomers and isomers of compounds with more than one chiral center that are not mirror images of one another (i.e., “diastereoisomers”).

“Improved enzymatic properties” refers to an improved hydantoinase polypeptide showing any enzymatic properties compared to a reference hydantoinase, such as a wild-type hydantoinase or another improved engineered hydantoinase. Desired improved enzyme properties include, but are not limited to, enzyme activity (which can be expressed as a percentage conversion of the substrate), thermal stability, solvent stability, pH activity characteristics, tolerance to inhibitors (e.g., substrate or product inhibition), and stereoselectivity.

“Conversion” refers to the enzymatic transformation of the substrate to the corresponding product. “Percent conversion” or “conversion” refers to the percentage of substrate that is converted to product within a period of time under the specified conditions. Thus, “enzymatic activity” or “activity” of a hydantoinase peptide can be expressed as the “percent conversion” of the substrate to the product. The conversion rate is generally calculated by sampling to measure the concentration of product and substrate in the reaction system: {molar concentration of product}/{molar concentration of substrate+molar concentration of product}.

“Thermostable” means that the hydantoinase polypeptide maintains similar activity after exposure to elevated temperatures (e.g., 72° C. or higher) for a sustained period of time (e.g., 2.5 hours or longer) compared to the wild-type enzyme.

“Solvent stable” or “solvent tolerant” means that the hydantoinase polypeptide maintains similar activity after exposure to different concentrations (e.g., 5-99%) of solvents (methanol, ethanol, isopropanol, dimethyl sulfoxide (DMSO), tetrahydrofuran, 2-Methyltetrahydrofuran, acetone, toluene, butyl acetate, methyl tert-butyl ether, etc.) for a period of time (e.g., 0.5-24 hours) compared to the wild-type enzyme.

“Suitable reaction conditions” refers to those conditions (e.g., enzyme loading, substrate loading, temperature, pH, buffer, cosolvent, etc.) in the biocatalytic reaction system, under which the hydantoinase polypeptide of the present disclosure converts the substrate to the desired product compound. Exemplary “suitable reaction conditions” are provided in the present disclosure and illustrated by examples.

“Hydrocarbyl” refers to a straight or branched hydrocarbon group. The number of subscripts following the symbol “C” specifies the number of carbon atoms that a particular group may contain. For example, “C₁-C₈” refers to a straight or branched chain hydrocarbyl group having 1 to 8 carbon atoms. Hydrocarbyl groups may optionally be substituted with one or more substituent groups. “Aryl” means a monovalent aromatic hydrocarbon radical of 6 to about 20 carbon atoms. “Heteroaryl” and “Heteroaryl” and “heteroaromatic” refer to an aryl group in which one or more of the carbon atoms of the parent aromatic ring system is/are replaced by a heteroatom (O, N, or S). “Substituted”, when used to modify a specified group or radical, means that one or more hydrogen atoms of the specified group or radical are each replaced, independently of one another, by identical or different substituents.

“Substituted hydrocarbyl, aryl, or heteroaryl” refers to a hydrocarbyl, aryl, or heteroaryl group in which one or more hydrogen atoms are replaced by other substituents. “Optional” or “optionally” means that the described event or circumstance may or may not occur; for example, “optionally substituted aryl” refers to an aryl group that may or may not be substituted. This description includes both substituted aryl groups and unsubstituted aryl groups.

The term “compound” refers to any compound encompassed by the structural formulas and/or chemical names indicated with the compounds disclosed herein. Compounds may be identified by their chemical structure and/or chemical name. When the chemical structure and chemical name conflict, the chemical structure determines the identity of the compound. Unless specifically stated or indicated otherwise, the chemical structures described herein encompass all possible isomeric forms of the described compounds.

### 2. Engineered Hydantoinase Peptides

The engineered polypeptide disclosed in the present invention has been developed from a wild-type hydantoinase through a creative process of directed evolution with a certain number of amino acid residue substitutions, insertions or deletions; the description of the directed evolution technique can be found in “Directed Evolution: Bringing New Chemistry Frances H. Arnold, Angewandte Chemie, Nov. 28, 2017. Frances H. Arnold was awarded the 2018 Nobel Prize in Chemistry for her pioneering contributions to the technology of directed evolution of enzymes. The wild-type hydantoinase is from Pseudomonas fluorescens and its amino acid sequence is shown in SEQ ID NO: 2. As tested by the inventors, the wild-type hydantoinase corresponding to SEQ ID NO: 2 shows poor activity on A1, which is greatly influenced by pH; moreover, this enzyme has poor tolerance to high concentration of product A2 and shows poor thermal stability. These defects are not conducive to industrial application, and SEQ ID NO: 2 needs to be engineered through directed evolution.

The protein corresponding to SEQ ID NO:2 has no publicly available 3D structure. The inventors used Yasara software to construct its 3D structure model, and then combined with bioinformatics techniques to design site-directed saturation mutagenesis libraries or multi-site combinatorial mutagenesis libraries for multiple residues. These libraries were then screened at different stages of development using the screening assay conditions shown in Tables 1.1, 2.1, 2.2, and Tables 3.1-3.4, respectively. Mutagenic libraries can be constructed using site-directed mutagenesis PCR (as shown in Example 2) or multi-site mutagenesis PCR (refer to “Mutagenesis and Synthesis of Novel Recombinant Genes Using PCR,” Chapter 32, in PCR Primer, 2nd edition (eds. Dieffenbach and Dveksler. Cold Spring Harbor Laboratory Press, Cold Spring Harbor, NY, USA, 2003).

In order to develop enzyme catalysts with excellent performance for the reaction shown in FIG. 5, the present invention carried out directed evolution of SEQ ID NO: 2 in several stages, with different high-throughput screening assay conditions designed for the different properties of the enzymes to be improved. The first stage was mainly for the improvement of the enzyme activity, and the designed high-throughput screening assay conditions are shown in Table 1.1 or Example 8. Some exemplary engineered polypeptides obtained in the first stage and their screening results are listed in Table 1.

TABLE 1
Exemplary engineered polypeptides obtained in the
first stage of directed evolution

| SEQ | Amino acid | Sequence | Activity fold from |
|---|---|---|---|
| ID NO: | residue | identity | high-throughput |
| (DNA\| \| | differences | to | screening assays |
| Amino | relative to SEQ | SEQ ID | (relative to SEQ ID |
| acid) | ID NO: 2 | NO: 2 | NO: 2) |
| 1\| \|2 | — | 100.0% | 1 |
| 3\| \|4 | M62L; | 99.7% | 1.8 |
| 5\| \|6 | Q63E; | 99.7% | 2.1 |
| 7\| \|8 | L64I; | 99.7% | 2.1 |
| 9\| \|10 | L64T; | 99.7% | 6.0 |
| 11\| \|12 | L64S; | 99.7% | 5.6 |
| 13\| \|14 | L64A; | 99.7% | 2.0 |
| 15\| \|16 | F66Y; | 99.7% | 1.8 |
| 17\| \|18 | F66L; | 99.7% | 1.9 |
| 19\| \|20 | M67W; | 99.7% | 1.9 |
| 21\| \|22 | M67Y; | 99.7% | 2.6 |
| 23\| \|24 | M67F; | 99.7% | 6.2 |
| 25\| \|26 | A71T; | 99.7% | 2.0 |
| 27\| \|28 | N97G; | 99.7% | 1.8 |
| 29\| \|30 | N97D; | 99.7% | 1.7 |
| 31\| \|32 | N97L; | 99.7% | 1.9 |
| 33\| \|34 | I159L; | 99.7% | 1.9 |
| 35\| \|36 | I159F; | 99.7% | 2.0 |
| 37\| \|38 | F320S; | 99.7% | 2.4 |
| 39\| \|40 | F320L; | 99.7% | 2.0 |
| 41\| \|42 | P336M; | 99.7% | 2.5 |
| 43\| \|44 | P336L; | 99.7% | 2.4 |
| 45\| \|46 | P336Q; | 99.7% | 2.4 |
| 47\| \|48 | N337P; | 99.7% | 3.0 |

TABLE 1
Screening assay condition of the first
stage (for catalytic activity)

| Enzyme concentration | 10 | g/L |
|---|---|---|
| Loading of substrate A1 | 2 | g/L |
| DMSO concentration | 10% | |
| PBS buffer concentration | 0.05M | |
| reaction pH | pH7.0 | |
| temperature | 30° | C. |
| Reaction time | 22 | h |

Notes:
1. In the screening reaction, DMSO is the co-solvent of substrate A1. The solubility of 3-isobutylglutarimide in aqueous solution is extremely low. In order to facilitate the preparation of the screening reaction in 96-well plates, the substrate needs to be dissolved in pure DMSO to prepare the stock solution first, and then mixed with other reaction components to complete the preparation of the screening reaction.
2. the conversion of substrate A1 by SEQ ID No: 2 is about 12.5% under this screening assay condition.
3. the activity fold of each engineered polypeptide relative to SEQ ID NO: 2 shown in Table 1 is the ratio obtained by dividing the conversion result of the engineered polypeptide by the conversion result of SEQ ID NO: 2. The sequences listed in Table 1 were preferentially selected from the variants in the first stage study according to the screening conditions in Table 1.1.

In practical industrial applications, the simpler the reaction system, the better. Generally, no co-solvents such as DMSO are used, and the substrate loading should be as high as possible. In order to test the catalytic effect of the engineered polypeptides shown in Table 1 under conditions relevant to industrial applications and to compare it with the wild-type SEQ ID NO: 2, the exemplary engineered polypeptides obtained in the first stage was tested using the following reaction conditions: the loading of substrate A1 was 10 g/L, and the loading of wet cells expressing engineered polypeptides was 50 g/L, 0.1M PBS pH7.0, 30° C. The reaction procedure is described as in Example 12. The results are shown in Table 1.2.

TABLE 1.2
Catalytic effect of the engineered polypeptides
from first-stage under reaction
conditions relevant to industrial applications

| | Concentration of | The ee value of |
|---|---|---|
| | product A2 after | product A2 after |
| SEQ ID NO: | 24 hours of | 24 hours of |
| (amino acid) | reaction | reaction |
| 2 | 1.2 g/L | 97% |
| 4, 6, 8, 10, 12, 14, 16, | 2.0-8.6 gL | 97%-99.9% |
| 18, 20, 22, 24, 26, | | |
| 28, 30, 32, 34, 36, 38, | | |
| 40, 42, 44, 46, 48 | | |

In the second stage, the directed evolution of improving pH stability was added along with the improvement of enzyme activity. The designed high-throughput screening assay conditions are shown in Table 2.1 and Table 2.2. Some exemplary engineered polypeptides obtained in the second stage and their screening results are listed in Table 2.

TABLE 2
Exemplary engineered polypeptides obtained in the second stage of
directed evolution

| | | | Catalytic | |
|---|---|---|---|---|
| | | | performance | |
| | | | fold as | |
| | | | determined | |
| | | | by high | |
| SEQ ID | | | throughput | pH |
| NO: | | Sequence | screening | stability |
| (DNA\| \| | Amino acid residue | identity | (relative to | (see note |
| Amino | differences relative to | with SEQ | SEQ ID NO: | in Table |
| acid) | SEQ ID NO: 2 | ID NO: 2 | 2) | 2.2.) |
| 1\| \|2 | — | 100% | 1 | * |
| 49\| \|50 | L64T; I159Y; | 99.5% | 24.0 | ** |
| 51\| \|52 | L64T; I159F; | 99.5% | 11.5 | *** |
| 53\| \|54 | L64T; I159L; | 99.5% | 10.6 | *** |
| 55\| \|56 | L64T; L189I; | 99.5% | 9.3 | ** |
| 57\| \|58 | L64T; L189V; | 99.5% | 8.8 | ** |
| 59\| \|60 | L64T; G201H; | 99.5% | 11.1 | ** |
| 61\| \|62 | L64T; Q215A; | 99.5% | 10.8 | ** |
| 63\| \|64 | L64T; Q215P; | 99.5% | 8.0 | ** |
| 65\| \|66 | L64T; S254Q; | 99.5% | 16.6 | ** |
| 67\| \|68 | L64T; S254L; | 99.5% | 19.5 | ** |
| 69\| \|70 | L64T; S254N; | 99.5% | 20.4 | ** |
| 71\| \|72 | L64T; S254G; | 99.5% | 14.3 | ** |
| 73\| \|74 | L64T; S254F; | 99.5% | 15.9 | ** |
| 75\| \|76 | L64T; K255F; | 99.5% | 14.3 | ** |
| 77\| \|78 | L64T; K255Y; | 99.5% | 10.6 | ** |
| 79\| \|80 | L64T; K255H; | 99.5% | 12.5 | ** |
| 81\| \|82 | L64T; K255N; | 99.5% | 17.0 | ** |
| 83\| \|84 | L64T; Q257W; | 99.5% | 18.2 | ** |
| 85\| \|86 | L64T; R329A; | 99.5% | 9.7 | ** |
| 87\| \|88 | L64T; R329L; | 99.5% | 11.1 | ** |
| 89\| \|90 | L64T; R329Y; | 99.5% | 10.6 | ** |
| 91\| \|92 | L64T; P474W; | 99.5% | 10.6 | ** |
| 93\| \|94 | L64T; A476P; | 99.5% | 7.8 | ** |
| 95\| \|96 | L64T; R479Q; | 99.5% | 7.5 | * |
| 97\| \|98 | L64T; R479L; | 99.5% | 7.6 | * |
| 99\| \|100 | L64T; R479P; | 99.5% | 7.4 | * |
| 101\| \|102 | L64T; A71T; N337P; | 99.3% | 12.3 | ** |
| 103\| \|104 | L64T; F66Y; N97L; I159F; N337P; | 98.9% | 12.0 | *** |
| 105\| \|106 | L64T; N97L; I159F; | 99.3% | 12.7 | ** |
| 107\| \|108 | L64T; M67F; | 99.5% | 9.9 | *** |
| 109\| \|110 | L64T; N337P; | 99.5% | 10.4 | *** |
| 111\| \|112 | L64T; F152Y; I159F; | 99.3% | 9.1 | ** |
| 113\| \|114 | L64T; I95V; I159F; | 99.3% | 7.9 | ** |
| 115\| \|116 | L64T; V263T; L264C; A265P; M288C; F292L; | 98.7% | 7.6 | *** |
| 117\| \|118 | L64T; L264C; A265P; G266Q; H267Y; M288C; | 98.7% | 13.0 | *** |
| 119\| \|120 | L64T; V263T; A265P; H267Y; F292L; | 98.9% | 11.8 | *** |
| 121\| \|122 | L51V; L64T; A340P; | 99.3% | 7.6 | ** |
| 123\| \|124 | A8G; G46A; L51V; L64T; A340P; | 98.9% | 11.5 | ** |
| 125\| \|126 | L64T; A340P; | 99.5% | 9.6 | ** |
| 127\| \|128 | L51I; L64T; A340P; | 99.3% | 13.5 | ** |
| 129\| \|130 | A8G; L51I; L64T; | 99.3% | 7.6 | ** |
| 131\| \|132 | G46A; L51I; L64T; A340P; | 99.1% | 10.6 | ** |
| 133\| \|134 | A8G; G46A; L51V; L64T; E73D; | 98.9% | 10.4 | ** |
| 135\| \|136 | A8G; L64T; A340P; | 99.3% | 11.5 | ** |
| 137\| \|138 | A8G; G46A; L51V; L64T; | 99.1% | 9.5 | ** |
| 139\| \|140 | G46A; L64T; A340P; | 99.3% | 8.3 | ** |
| 141\| \|142 | A8G; G46A; L51I; L64T; A340P; | 98.9% | 10.0 | ** |
| 143\| \|144 | G46A; L51V; L64T; A340P; | 99.1% | 15.5 | ** |
| 145\| \|146 | A8G; G46A; L64T; A340P; | 99.1% | 13.4 | ** |
| 147\| \|148 | L641; M67F; A71S; | 99.3% | 8.4 | ** |
| 149\| \|150 | L64T; F66Y; M67F; A71T; | 99.1% | 2.6 | *** |
| 151\| \|152 | L64I; M67Y; A71S; | 99.3% | 6.9 | *** |
| 153\| \|154 | L64I; M67W; A71S; | 99.3% | 4.4 | *** |
| 155\| \|156 | L64I; M67F; | 99.5% | 7.2 | ** |
| 157\| \|158 | L64I; M67Y; | 99.5% | 7.1 | ** |
| 159\| \|160 | L64S; F66Y; M67F; N97L; | 99.1% | 3.3 | *** |
| 161\| \|162 | L64T; N97L; I159F; N337P; | 99.1% | 22.2 | *** |
| 163\| \|164 | L64T; F66Y; A71T; I159F; N337P; | 98.9% | 10.6 | *** |
| 165\| \|166 | L64T; F66Y; I159F; N337P; | 99.1% | 21.9 | ** |
| 167\| \|168 | L64S; F66Y; M67F; A71T; | 99.1% | 3.1 | *** |
| 169\| \|170 | L64T; I159F; N337P; | 99.3% | 18.4 | ** |
| 171\| \|172 | L64T; I159F; N337P; K467D; | 99.1% | 7.5 | *** |
| 173\| \|174 | L64T; I159F; N337P; F462R; | 99.1% | 7.2 | *** |
| 175\| \|176 | L64T; I95L; F152M; I159F; | 99.1% | 2.6 | ** |
| 177\| \|178 | L64T; F152L; I159F; | 99.3% | 9.2 | ** |
| 179\| \|180 | L64T; I95M; I159F; | 99.3% | 9.4 | ** |
| 181\| \|182 | L64T; I95L; I159F; | 99.3% | 9.9 | ** |

TABLE 2.1
Screening assay condition for the second
stage (for catalytic activity)

| Enzyme concentration | 3 | g/L |
|---|---|---|
| Loading of substrate A1 | 2 | g/L |
| DMSO concentration | 10% | |
| PBS buffer concentration | 0.05M | |
| reaction pH | pH 7.0 | |
| temperature | 30° | C. |
| Reaction time | 22 | h |

Notes:
1. the conversion of substrate A1 by SEQ ID No: 2 was approximately 3.5% under the condition of this screening reaction.
2. The activity fold of each engineered polypeptide shown in Table 2 relative to SEQ ID No: 2 is the ratio obtained by dividing the conversion result of the engineered polypeptide by the conversion result of SEQ ID No: 2. The sequences listed in Table 2 were preferentially selected from the variants in the second stage study according to the screening conditions in Tables 2.1-2.2.

TABLE 2.2
Screening assay condition for the
second stage (for pH stability)

| Enzyme solution | pH 6.3, shaking at room | |
|---|---|---|
| pretreatment | temperature for 23 hours | |
| Enzyme concentration | 3 | g/L |
| Loading of substrate A1 | 2 | g/L |
| DMSO concentration | 10% | |
| PBS buffer concentration | 0.05M | |
| reaction pH | pH 7.0 | |
| temperature | 30° | C. |
| Reaction time | 22 | h |

Notes:
1. The method of enzyme solution pretreatment is referred to Example 9.
2. The pH stability of each polypeptide shown in Table 2 is graded according to the ratio of its conversion measured under the conditions of Table 2.2 to that measured under the conditions of Table 2.1: a ratio <50% is “*”, a ratio between 50% and 80% is “*”, ratio > 80% as “*”.
3. Under this screening reaction condition, the conversion of A1 by SEQ ID NO: 2 after [pH 6.3, 23 h] pretreatment was ~1%, and the ratio of 1%/3.5% was in the interval of <50%; therefore, its pH stability is in the “*” grade.

The exemplary engineered polypeptide obtained in the second stage was assayed using the following reaction conditions: a load of 10 g/L of substrate A1, a load of 6 g/L of wet cells expressing the engineered polypeptides, 0.1 M PBS pH 7.0, and 30° C. The reaction procedure was as described in Example 13. The results are shown in Table 2.3.

TABLE 2.3
Catalytic effect of the engineered polypeptides from the second stage
under reaction conditions relevant to industrial applications

| | Concentration | The ee |
|---|---|---|
| | of | value of |
| | product | product |
| | A2 after | A2 after |
| SEQ ID NO: | 24 hours of | 24 hours of |
| (amino acid) | reaction | reaction |
| 2 | 0.15 g/L | 97% |
| 50, 52, 54, 56, 58, 60, 62, 64, 66, 68, 70, | 5.0-9.5 g/L | 97%-99.9% |
| 72, 74, 76, 78, 80, 82, 84, 86, 88, 90, 92, | | |
| 94, 96, 98, 100, 102, 104, 106, 108, 110, 112, | | |
| 114, 116, 118, 120, 122, 124, 126, 128, 130, | | |
| 132, 134, 136, 138, 140, 142, 144, 146, 148, | | |
| 150, 152, 154, 156, 158, 160, 162, 164, 166, | | |
| 168, 170, 172, 174, 176, 178, 180, 182 | | |

In the third stage of directed evolution, on top of improving enzyme activity and pH stability, it further aimed for improving the tolerance towards high-concentration product and improving thermal stability. The designed high-throughput screening assay conditions are shown in Table 3.1, Table 3.2, Table 3.3 and Table 3.4. Some exemplary engineered polypeptides obtained in the third stage and their screening reaction results are listed in Tables 3 and 3.5.

TABLE 3
Exemplary engineered polypeptides obtained in the third stage of directed
evolution

| | | | Activity of |
|---|---|---|---|
| | | | the high- |
| | | | through- |
| SEQ | | | put |
| ID NO: | | Sequence | screening |
| (DNA\| \| | | identity | assay |
| amino | | with SEQ | (Table |
| acid) | Amino acid residue differences relative to SEQ ID NO: 2 | ID NO: 2 | 3.1) |
| 1\| \|2 | — | 100% | NA |
| 183\| \|184 | L51I; L64T; F66Y; A71T; N97L; I159L; L189V; Q215A; N337P; | 97.9% | ++ |
| | A340P; | | |
| 185\| \|186 | L51I; L64T; A71T; I159L; L189I; Q215A; N337P; A340P; | 98.3% | +++ |
| 187\| \|188 | L51I; L64T; F66Y; N97L; I159Y; Q215A; N337P; A340P; | 98.3% | ++ |
| 189\| \|190 | L51I; L64T; I159Y; L189V; Q215A; N337P; A340P; | 98.5% | ++ |
| 191\| \|192 | L51I; L64T; N97Q; I159F; N337P; A340P; | 98.7% | ++ |
| 193\| \|194 | L51I; L64T; A71T; N97L; I159L; L189V; Q215A; N337P; A340P; | 98.1% | ++ |
| 195\| \|196 | L51I; L64T; F66Y; A71T; I159Y; L189V; Q215A; N337P; A340P; | 98.1% | ++ |
| 197\| \|198 | L51I; L64T; A71T; N97L; I159L; L189I; Q215A; N337P; A340P; | 98.1% | ++ |
| 199\| \|200 | L51I; L64T; A71T; I159F; Q215A; N337P; A340P; | 98.5% | ++ |
| 201\| \|202 | L51I; L64T; A71T; I159Y; L189I; Q215A; N337P; A340P; | 98.3% | ++ |
| 203\| \|204 | A39P; L51I; L64T; A71T; I159L; L189I; Q215A; N337P; A340P; | 98.1% | ++ |
| 205\| \|206 | L51I; L64T; F66Y; A71T; N97L; I159L; Q215A; N337P; A340P; | 98.1% | ++ |
| 207\| \|208 | L51I; L64T; F66Y; A71T; I159L; L189I; Q215A; N337P; A340P; | 98.1% | ++ |
| 209\| \|210 | L51I; L64T; F66Y; N97L; 159F; L189I; Q215A; N337P; A340P; | 98.1% | ++ |
| 211\| \|212 | L51I; L64T; A71T; I159L; L189V; N337P; | 98.7% | ++ |
| 213\| \|214 | L51I; L64T; F66Y; A71T; I159F; Q215A; N337P; A340P; | 98.3% | ++ |
| 215\| \|216 | L51I; L64T; F66Y; A71T; I159F; L189V; Q215A; N337P; A340P; | 98.1% | ++ |
| 217\| \|218 | L51I; L64T; F66Y; I159Y; L189; Q215A; N337P; A340P; | 98.3% | ++ |
| 219\| \|220 | L51I; L64T; A71T; A113T; I159L; L189V; Q215A; N337P; A340P; | 98.1% | + |
| 221\| \|222 | L51I; L64T; A71T; I159L; L189V; Q215A; N337P; A340P; | 98.3% | ++ |
| 223\| \|224 | L51I; L64T; A71T; I159Y; L189V; Q215A; N337P; A340P; | 98.3% | ++ |
| 225\| \|226 | L51I; L64T; F66Y; I159F; L189I; Q215A; N337P; A340P; | 98.3% | ++ |
| 227\| \|228 | L51I; L64T; F66Y; A71T; I159F; L189I; Q215A; N337P; A340P; | 98.1% | ++ |
| 229\| \|230 | L51I; L64T; A71T; N97L; I159F; N337P; A340P; | 98.5% | + |
| 231\| \|232 | L51I; L64T; N97L; I159Y; L189I; Q215A; N337P; A340P; | 98.3% | ++ |
| 233\| \|234 | L51I; L64T; F66Y; N97L; I159Y; L189I Q215A; N337P; A340P; | 98.1% | ++ |
| 235\| \|236 | L51I; L64T; A71T; I159F; L189V; Q215A; N337P; A340P; | 98.3% | ++ |
| 237\| \|238 | L51I; L64T; I159F; L189I; Q215A; N337P; A340P; | 98.5% | +++ |
| 239\| \|240 | L51I; L64T; F66Y; I159F; Q215A; N337P; A340P; | 98.5% | +++ |
| 241\| \|242 | L64T; I159F; L189V; Q215A; K255H; Q257W; N337P; | 98.5% | +++ |
| 243\| \|244 | L64T; I159F; L189V; Q215A; K255N; Q257W; N337P; A340P; | 98.3% | +++ |
| 245\| \|246 | L64T; I159F; L189I; Q215A; K255N; N337P; | 98.7% | + |
| 247\| \|248 | L51I; L64T; I159F; L189V; Q215A; K255H; N337P; | 98.5% | +++ |
| 249\| \|250 | L64T; I159F; L189V; Q215A; K255N; Q257W; N337P; | 98.5% | +++ |
| 251\| \|252 | L64T; I159F; L189I; Q215A; K255H; Q257W; N337P; | 98.5% | +++ |
| 253\| \|254 | L64T; I159F; L189I; Q215P; K255H; Q257W; N337P; | 98.5% | +++ |
| 255\| \|256 | L64T; I159F; L189I; Q215A; K255H; N337P; | 98.7% | +++ |
| 257\| \|258 | L64T; I159F; L189I; A199V; Q215A; K255N; N337P; | 98.5% | + |
| 259\| \|260 | L64T; I159F; L189I; Q215P; K255N; Q257W; N337P; | 98.5% | +++ |
| 261\| \|262 | L64T; I159F; L189V; Q215P; K255N; Q257W; N337P; | 98.5% | +++ |
| 263\| \|264 | L64T; I159F; L189I; Q215A; K255N; Q257W; N337P; | 98.5% | +++ |
| 265\| \|266 | L64T; I159F; L189I; Q215P; K255H; Q257W; N337P; A340P; | 98.3% | +++ |
| 267\| \|268 | L51I; L64T; I159F; L189I; Q215P; K255H; Q257W; N337P; | 98.3% | +++ |
| 269\| \|270 | L64T; I159F; L189V; Q215A; K255H; N337P; | 98.7% | +++ |
| 271\| \|272 | L51I; L64T; I159F; L189V; Q215P; K255H; Q257W; N337P; | 98.3% | +++ |
| 273\| \|274 | L51I; L64T; I159F; L189V; Q215P; N337P; | 98.7% | ++ |
| 275\| \|276 | L51I; L64T; I159F; L189V; Q215A; K255H; Q257W; N337P; | 98.3% | +++ |
| 277\| \|278 | L64T; I159F; L189I; Q215P; K255N; N337P; | 98.7% | + |
| 279\| \|280 | L64T; I159F; L189I; Q215P; N337P; | 98.9% | +++ |
| 281\| \|282 | L64T; I159F; L189M; Q215P; K255H; Q257W; N337P; | 98.5% | +++ |
| 283\| \|284 | L64T; I159F; Q215P; K255H; Q257W; N337P; | 98.7% | +++ |
| 285\| \|286 | L51I; L64T; I159F; L189I; Q215P; K255H; N337P; | 98.5% | ++ |

TABLE 3.1
Screening assay condition for the
third stage (for catalytic activity)

| Enzyme concentration | 0.3 | g/L |
|---|---|---|
| Loading of substrate A1 | 2 | g/L |
| DMSO concentration | 10% | |
| PBS buffer concentration | 0.05M | |
| reaction pH | pH 7.0 | |
| temperature | 30° | C. |
| Reaction time | 22 | h |

Notes:
1. Due to the existence of baseline noise in the HPLC assay, the minimum detection limit of A1 conversion for this screening reaction condition was 0.5%. Under this screening reaction condition, the concentration of enzyme solution containing SEQ ID NO: 2 was reduced to 0.3 g/L, and its detection result for A1 conversion was < 0.5%. Therefore, its catalytic activity could not be accurately determined, as the conversion of A1 by SEQ ID NO: 2 was already below the lower detection limit.
2. The conversion result of each engineered polypeptide shown in Table 3 was graded according to its conversion of A1 measured under the condition of Table 3.1: conversion between 0.5%-30% is “+”, conversion between 30%-50% is “++”, and “++” for a conversion > 50%. The sequences listed in Table 3 were preferentially selected from all the variants in the third stage study according to the screening conditions in Tables 3.1-3.4.

TABLE 3.2
Screening assay conditions for
the third stage (for pH stability)

| | pH6.3, Shake at room | |
|---|---|---|
| Enzyme pretreatment | temperature for 23 hours | |
| Loading of substrate A1 | 0.3 | g/L |
| DMSO concentration | 2 | g/L |
| PBS buffer concentration | 10% | |
| reaction pH | 0.05M | |
| temperature | pH 7.0 | |
| Reaction time | 30° | C. |
| Loading of substrate A1 | 22 | h |

Notes:
1. The pH stability of each engineered polypeptide shown in Table 3.5 is graded according to the ratio of its conversion measured in Table 3.2 to that measured in Table 3.1: the ratio < 50% is “A”, the ratio between 50% and 80% is “AA”, the ratio > 80% is “AAA”.

TABLE 3.3
Screening assay condition for the
third stage (for product tolerance)

| Enzyme concentration | 0.3 | g/L |
|---|---|---|
| Loading of substrate A2 | 50 | g/L |
| Loading of substrate A1 (3- | 2 | g/L |
| isobutylglutarimide) | | |
| DMSO concentration | 10% | |
| PBS buffer concentration | 0.05M | |
| reaction pH | pH 7.0 | |
| temperature | 30° | C. |
| Reaction time | 22 | h |

Notes:
1. the screening reaction for product tolerance refers to Example 10.
2. The product tolerance of each engineered polypeptide shown in Table 3.5 is graded according to the ratio of its conversion measured under Table 3.3 conditions to that measured under Table 3.1 conditions: a ratio < 50% is “B”, a ratio between 50% and 80% is “BB”, the ratio > 80% is “BBB” .

TABLE 3.4
Screening assay condition for the
third stage (for thermal stability)

| Enzyme pretreatment | 50° C. water bath for 23 hours | |
|---|---|---|
| Enzyme concentration | 0.3 | g/L |
| Loading of substrate A1 | 2 | g/L |
| (3-isobutylglutarimide) | | |
| DMSO concentration | 10% | |
| PBS Buffer concentration | 0.05M | |
| reaction pH | pH 7.0 | |
| temperature reflex | 30° | C. |
| Reaction time | 22 | h |

Notes:
1. The screening reaction for thermal stability refers to Example 11.
2. The thermal stability of each engineered polypeptide shown in Table 3.5 is graded according to the ratio of its conversion measured under the conditions of Table 3.4 to that measured under the conditions of Table 3.1: a ratio < 50% is “C”, a ratio between 50% and 80% is “CC”, the ratio > 80% is “CCC”.

TABLE 3.5

| SEQ ID | pH stability | Product tolerance | Thermal |
|---|---|---|---|
| NO: | testing | testing | stability testing |
| 2 | NA | NA | NA |
| 184 | AAA | BBB | CC |
| 186 | A | BB | CC |
| 188 | AA | BBB | C |
| 190 | AA | BBB | C |
| 192 | AA | BBB | CC |
| 194 | AA | BBB | C |
| 196 | AA | BBB | C |
| 198 | AAA | BB | CC |
| 200 | AA | BBB | C |
| 202 | A | BBB | C |
| 204 | AAA | BBB | C |
| 206 | AAA | BBB | CC |
| 208 | AA | BBB | C |
| 210 | AAA | BBB | C |
| 212 | AA | B | CC |
| 214 | AAA | BBB | CCC |
| 216 | AA | BBB | CC |
| 218 | AA | BB | C |
| 220 | AA | BB | CC |
| 222 | AA | BBB | CC |
| 224 | AA | BBB | C |
| 226 | AAA | BBB | C |
| 228 | AA | BBB | C |
| 230 | AA | BBB | C |
| 232 | AA | BB | C |
| 234 | AAA | BBB | CC |
| 236 | AA | BBB | C |
| 238 | AA | BBB | C |
| 240 | AA | BBB | C |
| 242 | AAA | BB | C |
| 244 | AAA | BBB | CC |
| 246 | AAA | BBB | CC |
| 248 | AAA | BBB | CC |
| 250 | AAA | BBB | C |
| 252 | AAA | BBB | CC |
| 254 | A | BBB | C |
| 256 | AAA | BBB | C |
| 258 | A | BBB | C |
| 260 | AAA | BBB | C |
| 262 | AA | BBB | C |
| 264 | A | B | C |
| 266 | A | BB | C |
| 268 | AAA | BBB | C |
| 270 | AAA | BBB | C |
| 272 | AAA | BBB | C |
| 274 | A | BBB | C |
| 276 | AA | BBB | C |
| 278 | A | BBB | C |
| 280 | AA | BB | CCC |
| 282 | AA | BBB | C |
| 284 | AA | BBB | C |
| 286 | AA | BBB | CCC |

The exemplary engineered polypeptides obtained in the third stage were assayed using the following reaction conditions: a load of 10 g/L of substrate A1, a load of 1 g/L of wet cells expressing the engineered polypeptides, 0.1 M PBS pH 7.0, and 40° C. The reaction procedure was as described in Example 14. The results are shown in Table 3.6.

TABLE 3.6
Catalytic effect of the engineered polypeptides from the third stage under
reaction conditions relevant to industrial applications

| | Concentration of | The ee value of |
|---|---|---|
| | product A2 after | product A2 after |
| | 24 hours of | 24 hours of |
| SEQ ID NO: (amino acid) | reaction | reaction |
| 2 | A2 not detected | NA |
| 184, 186, 188, 190, 192, 194, 196, 198, 200, 202, 204, | | |
| 206. 208. 210. 212. 214, 216, 218, 220, 222, 224, 226, | | |
| 228. 230, 232. 234, 236, 238, 240, 242, 244, 246, 248, | 6-9.9 g/L | 97%-99.9% |
| 250. 252. 254, 256, 258, 260, 262, 264, 266, 268, 270, | | |
| 272, 274, 276, 278, 280, 282, 284, 286 | | |

Based on the properties of the exemplary engineered polypeptides listed in Tables 1, 2, and 3, the increase in enzymatic activity (i.e., conversion of compound A1 to compound A2) is associated with amino acid residue differences at the following residue positions as well as others: X8, X39, X46, X51, X62, X63, X64, X66, X67, X71, X73, X95, X97, X152, X159, X189, X199, X201, X215, X254, X255, X257, X263, X264, X265, X266, X267, X288, X292, X320, X329, X336, X337, X340, X462, X467, X474, X476, X479. In some embodiments, the amino acid residue differences compared to SEQ ID NO: 2 are selected from the group consisting of: A8G, A39P, G46A, L51V, L51I, M62L, Q63E, L64I, L64T, L64S, L64A, F66Y, F66L, M67W, M67Y, M67F, A71T, A71S, E73D, 195V, 195L, 195M, N97G, N97D, N97L, N97Q, F152Y, F152M, F152L, I159L, I159F, I159Y, L189I, L189V, L189M, Q215, QP, G201H, S254Q, S254L, S254N, S254G, S254F, K255F, K255Y, K255H, K255N, Q257W, V263T, L264C, A265P, G266Q, H267Y, M288C, F292L, F320S, F320L, R329A, R329L, R329Y, P336M, P336L, P336Q, N337P, A340P, F462R, K467D, P474W, A476P, R479Q, R479L, R479P.

Based on the properties of the exemplary engineered polypeptides listed in Table 2 and Table 3.5, the increase in enzyme's pH stability is correlated with amino acid residue differences at the following residue positions and others: X8, X39, X46, X51, X64, X66, X67, X71, X73, X95, X97, X113, X152, X159, X189, X201, X215, X254, X255, X257, X263, X264, X265, X266, X267, X288, X292, X329, X337, X340, X462, X467, X474, X476. In some embodiments, the amino acid residue differences compared to SEQ ID NO: 2 are selected from the group consisting of: A8G, A39P, G46A, L51V, L51I, L64T, L64I, L64S, F66Y, M67F, M67Y, M67W, A71T, A71S, E73D, 195V, 195L, 195M, N97L, N97Q, A113T, F152Y, F152M, F152L, I159Y, I159F, I159L, L189I, L189V, G201H, Q215A, Q215P, S254Q, S254L, S254, S254N, 255FK, K255H, K255N, Q257W, V263T, L264C, A265P, G266Q, H267Y, M288C, F292L, R329A, R329L, R329Y, N337P, A340P, F462R, K467D, P474W, A476P.

Based on the properties of the exemplary engineered polypeptides listed in Table 3.5, the increase in product tolerance and/or thermostability of the enzyme is associated with amino acid residue differences at the following residue positions as well as others: X39, X51, X64, X66, X71, X97, X113, X159, X189, X199, X215, X255, X257, X337, X340. In some embodiments, the amino acid residue differences compared to SEQ ID NO: 2 are selected from the group consisting of: A39P, L51I, L64T, F66Y, A71T, N97L, N97Q, A113T, I159L, I159Y, I159F, L189V, L189I, L189M, A199V, Q215A, Q215P, K255H, K255N, Q257W, N337P, A340P.

As will be apparent to those skilled in the art, the foregoing residue positions, and the specific amino acid residues at each residue position, can be used individually or in various combinations to give engineered hydantoinase polypeptides with desired properties, which include improved enzymatic activity, stereoselectivity, stability, and others.

Based on the guidance provided herein, it is further contemplated that any of the exemplary engineered polypeptides having even-numbered sequence identifiers in SEQ ID NOs: 4-286 can be used as starting amino acid sequences for the development of other engineered polypeptides, for example, by adding various amino acid differences from the residue positions described in Table 1, Table 2, and Table 3. Further improvements can be obtained by incorporating amino acid differences at positions that remain unchanged during the three stages of directed evolution described herein.

Thus, in some embodiments, an engineered polypeptide capable of converting compound A1 to compound A2 comprises, at least 90%, 91%, 92%, 93%, 94%, 95%, 96%, 97%, 98%, 99% or more identity with an even sequence identifier selected from the group consisting of SEQ ID NOs: 4-286, and compared to SEQ ID NO:2, the amino acid sequences having one or more residue differences at residue positions selected from: X8, X39, X46, X51, X62, X63, X64, X66, X67, X71, X73, X95, X97, X113, X152, X159, X189, X199, X201, X215, X254, X255, X257, X263, X264, X265, X266, X267, X288, X292, X320, X329, X336, X337, X340, X462, X467, X474, X476, X479.

In some embodiments, an engineered polypeptide capable of converting compound A1 to compound A2 under appropriate reaction conditions comprises, at least 90%, 91%, 92%, 93%, 94%, 95%, 96%, 97%, 98%, 99% or more identity with an even sequence identifier selected from the group consisting of SEQ ID NOs: 4-286, and compared to SEQ ID NO:2, the amino acid sequences having one or more residue differences at residue positions selected from: A8G, A39P, G46A, L51V, L51I, M62L, Q63E, L64I, L64T, L64S, L64A, F66Y, F66L, M67W, M67Y, M67F, A71T, A71S, E73D, 195V, 195L, 195M, N97G, N97D, N97L, N97Q, A113T, F152Y, F152M, F152L, I159L, I159F, I159Y, L189I, L189V, L189M, A199V, G201H, Q215A, Q215P, S254Q, S254L, S255F, K254N, S2K, K255H, K255N, Q257W, V263T, L264C, A265P, G266Q, H267Y, M288C, F292L, F320S, F320L, R329A, R329L, R329Y, P336M, P336L, P336Q, N337P, A340P, F462R, K467D, P474W, A476P, R479Q, R479L, R479P.

In addition to the residue positions specified above, any engineered polypeptide disclosed herein may also include residue positions at other residue positions, i.e., residue positions other than the following residue positions, relative to the reference polypeptide sequence of SEQ ID NO: 2: X8, X39, X46, X51, X62, X63, X64, X66, X67, X71, X73, X95, X97, X113, X152, X159, X189, X199, X201, X215, X254, X255, X257, X263, X264, X265, X266, X267, X288, X292, X320, X329, X336, X337, X340, X462, X467, X474, X476, X479. Residue differences at these other residue positions can provide additional variants in the amino acid sequence without altering the ability of the polypeptide to convert compound A1 to compound A2, particularly with respect to increased enzymatic activity, increased pH stability, increased product tolerance, as well as increased thermal stability. Thus, in some embodiments, in addition to amino acid residue differences in any of the engineered polypeptides selected from the polypeptides having the even-numbered sequence identifiers in SEQ ID NOs: 4-286, the sequence may also include 1, 2, 3, 4, 5, 6, 7, 8, 9, 10, 11, 12, 13, 14, 15, 16, 17, 18, 19, 20, 21, 22, 23, 24, or 25 residue differences at other amino acid residue positions compared to SEQ ID NO: 2.

### 3. Polynucleotides, Control Sequences, Expression Vectors and Host Cells that can be Used to Prepare Engineered Polypeptides

In another aspect, the present disclosure provides polynucleotides encoding the engineered polypeptides having hydantoinase activity described herein. The polynucleotides can be linked to one or more heterologous regulatory sequences that control gene expression to produce recombinant polynucleotides that are capable of expressing the engineered polypeptides. Expression constructs comprising a heterologous polynucleotide encoding an engineered hydantoinase may be introduced into a suitable host cell to express the corresponding engineered hydantoinase polypeptide.

As apparent to those skilled in the art, the availability of protein sequences and knowledge of codons corresponding to various amino acids provide an illustration of all possible polynucleotides that encode the protein sequence of interest. The degeneracy of the genetic code, in which the same amino acids are encoded by selectable or synonymous codons, allows for the production of an extremely large number of polynucleotides, all of which encode the engineered polypeptides disclosed herein. Thus, upon determination of a particular amino acid sequence, one skilled in the art can generate any number of different polynucleotides by merely modifying one or more codons in a manner that does not alter the amino acid sequence of the protein. In this regard, the present disclosure specifically contemplates each and every possible alteration of a polynucleotide that can be made by selecting combinations based on possible codon selections, for any of the polypeptides disclosed herein, comprising those amino acid sequences of exemplary engineered polypeptides listed in Table 1, Table 2 and Table 3, and any of the polypeptides disclosed as even sequence identifiers of SEQ ID NOS: 4 to 286 in the Sequence Listing incorporated by reference, all of which are believed to be particularly public.

In various embodiments, the codons are preferably selected to accommodate the host cell in which the recombinant protein is produced. For example, codons preferred for bacteria are used to express genes in bacteria; codons preferred for yeast are used to express genes in yeast; and codons preferred for mammals are used for gene expression in mammalian cells.

In some embodiments, the polynucleotides encode hydantoinase polypeptides comprising amino acid sequences that are at least about 90%, 91%, 92%, 93%, 94%, 95%, 96%, 97%, 98%, 99% or more identical to a reference sequence that is an even sequence identifier of SEQ ID NO: 4-286, wherein the polypeptides have hydantoinase activity and one or more of the improved properties described herein, for example, the ability to convert compound A1 to compound A2 with increased activity compared to the polypeptide of SEQ ID NO:2.

In some embodiments, the polynucleotides encode engineered polypeptides comprising amino acids sequences having a percentage of identity described above and having one or more amino acid residue differences as compared to SEQ ID NO: 2. In some embodiments, the present disclosure provides engineered polypeptides having hydantoinase activity, wherein the engineered polypeptides comprise a combination that has at least 90% sequence identity to the reference sequence of SEQ ID NO: 2 with residue differences that is selected from the following positions: X8, X39, X46, X51, X62, X63, X64, X66, X67, X71, X73, X95, X97, X113, X152, X159, X189, X199, X201, X215, X254, X255, X257, X263, X264, X265, X266, X267, X288, X292, X320, X329, X336, X337, X340, X462, X467, X474, X476, X479.

In some embodiments, the polynucleotides encoding the engineered polypeptides comprise a polynucleotide selected from SEQ ID No: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285.

In some embodiments, the polynucleotides encode polypeptides as described herein, but at the nucleotide level, the polynucleotides have about 50%, 55%, 60%, 65%, 70%, 75%, 80%, 85%, 86%, 87%, 88%, 89%, 90%, 91%, 92%, 93%, 94%, 95%, 96%, 97%, 98%, 99% or more sequence identity to reference polynucleotides encoding engineered hydantoinase polypeptides as described herein. In some embodiments, the reference polynucleotides are selected from SEQ ID No: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 8175, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271 sequences of 273, 275, 277, 279, 281, 283, 285.

The isolated polynucleotides encoding engineered polypeptides can be manipulated to enable the expression of the engineered polypeptides in a variety of ways, which comprises further modification of the sequences by codon optimization to improve expression, insertion into suitable expression elements with or without additional control sequences, and transformation into a host cell suitable for expression and production of the engineered polypeptides.

Depending on the expression vector, manipulation of the isolated polynucleotide prior to insertion of the isolated polynucleotide into the vector may be desirable or necessary. Techniques for modifying polynucleotides and nucleic acid sequences using recombinant DNA methods are well known in the art. Guidance is provided below: Sambrook et al., 2001, Molecular Cloning: A Laboratory Manual, Third Edition, Cold Spring Harbor Laboratory Press; and Current Protocols in Molecular Biology, Ausubel. F. Eds., Greene Pub. Associates, 1998, updated in 2010.

In another aspect, the present disclosure also relates to recombinant expression vectors, depending on the type of host they are to be introduced into, including a polynucleotide encoding an engineered polypeptide or variant thereof, and one or more expression regulatory regions, such as promoters and terminators, origin of replication and the like. Alternatively, the nucleic acid sequence of the present disclosure can be expressed by inserting the nucleic acid sequence or the nucleic acid construct comprising the sequence into an appropriate expression vector. In generating the expression vector, the coding sequence is located in the vector such that the coding sequence is linked to a suitable control sequence for expression.

The recombinant expression vector can be any vector (e.g., plasmid or virus) that can be conveniently used in recombinant DNA procedures and can result in the expression of a polynucleotide sequence. The choice of vector will generally depend on the compatibility of the vector with the host cells to be introduced into. The vector may be a linear or closed circular plasmid. The expression vector may be an autonomously replicating vector, i.e., a vector that exists as an extrachromosomal entity whose replication is independent of chromosomal replication such as plasmids, extrachromosomal elements, microchromosomes, or artificial chromosomes. The vector may contain any tools for ensuring self-copying. Alternatively, the vector may be a vector that, when introduced into a host cell, integrates into the genome and replicates with the chromosome into which it is integrated. Moreover, a single vector or plasmid or two or more vectors or plasmids that together comprise the total DNA to be introduced into the genome of the host cell may be used.

Many expression vectors useful to the embodiments of the present disclosure are commercially available. An exemplary expression vector can be prepared by inserting a polynucleotide encoding an engineered hydantoinase polypeptide to plasmid pACYC-Duet-1 (Novagen).

In another aspect, the present disclosure provides host cells comprising a polynucleotides encoding engineered hydantoinase polypeptides of the present disclosure. The polynucleotide is linked to one or more control sequences for expression of hydantoinase polypeptides in the host cell. Host cells for expression of polypeptides encoded by the expression vectors of the present disclosure are well known in the art, including, but not limited to, bacterial cells such as Escherichia coli, Arthrobacter spp. KNK168, Streptomyces and Salmonella typhimurium cells; fungal cells such as yeast cells (e.g., Saccharomyces cerevisiae or Pichia pastoris); insect cells such as Drosophila S2 and Spodoptera Sf9 cells; animal cells such as CHO, COS, BHK, 293 and Bowes melanoma cells; and plant cells. An exemplary host cells are E. coli BL21(DE3). The above host cells may be wild-type or may be engineered cells through genomic edition, such as knockout of the wild-type hydantoinase gene carried in the host cell's genome. Suitable media and growth conditions for the above host cells are well known in the art.

Polynucleotides used to express engineered hydantoinase can be introduced into cells by a variety of methods known in the art. Techniques comprise, among others, electroporation, bio-particle bombardment, liposome-mediated transfection, calcium chloride transfection, and protoplast fusion. Different methods of introducing polynucleotides into cells are obvious to those skilled in the art.

### 4. Process of Producing an Engineered Polypeptide

When the sequence of an engineered polypeptide is known, the encoding polynucleotide may be prepared by standard solid-phase methods according to known synthetic methods. In some embodiments, fragments of up to about 100 bases may be synthesized separately and then ligated (e.g., by enzymatic or chemical ligation methods or polymerase-mediated methods) to form any desired contiguous sequence. For example, the polynucleotides and oligonucleotides of the present disclosure may be prepared by chemical synthesis using, for example, the classic phosphoramidite methods described by Beaucage et al, 1981, TetLett22:1859-69, or Matthes et al., 1984, EMBOJ. 3:801-05, as typically practiced in automated synthetic methods. According to the phosphoramidite method, oligonucleotides are synthesized, purified, annealed, ligated, and cloned into a suitable vector, for example, in an automated DNA synthesizer. In addition, essentially any nucleic acid is available from any of a variety of commercial sources.

In some embodiments, the present disclosure also provides a process for preparing or producing an engineered polypeptide, wherein the process comprises culturing a host cell capable of expressing a polynucleotide encoding the engineered polypeptide under culture conditions suitable for expression of the polypeptide. In some embodiments, the process of preparing the polypeptide further comprises isolating the polypeptide. The engineered polypeptides may be expressed in suitable cells and isolated (or recovered) from the host cells and/or culture medium using any one or more of the well-known techniques for protein purification, the techniques for protein purification include, among others, lysozyme treatment, sonication, filtration, salting out, heat treatment, ultracentrifugation, and chromatography.

### 5. Methods of Using Engineered Hydantoinase and Compounds Prepared Therewith:

The present disclosure also provides processes for preparing the compounds of structural formula (I) using the engineered hydantoinase polypeptides described herein:

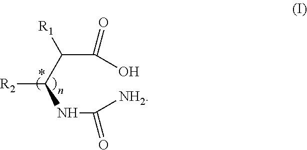

The compounds of structural formula (I) have the indicated stereochemical configuration at the chiral center marked with *; each of the compounds of structural formula (I) is in an enantiomeric excess over the other enantiomer, where n=0 or 1; R₁, R₂ are independently of each other selected from H, optionally substituted or unsubstituted aryl or heteroaryl, straight or branched and optionally substituted or unsubstituted C₁-C₄ alkyl, straight or branched and optionally substituted or unsubstituted C₁-C₄ alkenyl, optionally substituted or unsubstituted cycloalkyl, —OR′, —NH₂ or —NR′R′, —SR′, —CO₂R′, or —C(O)R′; wherein each R′ is independently selected from —H or (C₁-C₄) hydrocarbon groups.

The process herein comprises that, the hydantoin-derived substrate of formula (II),

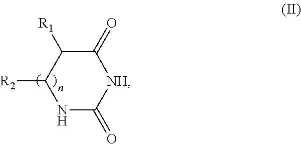

is contacted with the engineered hydantoinase polypeptide, the definitions of n, R₁, R₂ in said structural formula (II) are the same as in structural formula (I).

In another aspect, the present disclosure also provides processes for preparing the compounds of structural formula (III) using the engineered hydantoinase polypeptides described herein:

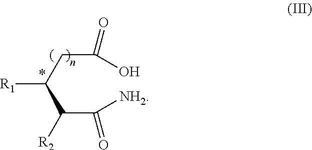

The compounds of said structural formula (III) have the indicated stereochemical configuration at the chiral center marked with *; each of the compounds of said structural formula (III) is in an enantiomeric excess over the other enantiomer, where n=0 or 1; R₁, R₂ are independently of each other selected from H, straight or branched and optionally substituted or unsubstituted C₁-C₄ alkyl, or optionally substituted or unsubstituted C₆H₆; when n=0, R₁, R₂ may also together form a ring structure group selected from monocyclic or polycyclic, optionally substituted or unsubstituted aryl groups or monocyclic or polycyclic, optionally substituted or unsubstituted heteroaryl groups.

The process herein comprises that, the substrate of formula (IV),

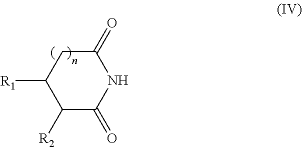

is contacted with the engineered hydantoinase polypeptide, the definitions of n, R₁, R₂ in said structural formula (IV) are the same as in structural formula (III).

In another aspect, the engineered polypeptide described herein converts DL-p-hydroxyphenylhydantoin to N-carbamoyl-D-p-hydroxyphenylglycine which is further converted to D-p-hydroxyphenylglycine in the presence of hydrochloric acid.

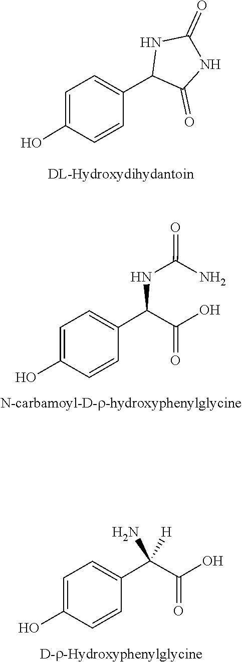 [SMILES: O=C1NC(=O)C(c2ccc(O)cc2)N1 ; NC(=O)N[C@@H](C(=O)O)c1ccc(O)cc1 ; N[C@@H](C(=O)O)c1ccc(O)cc1]

In another aspect, the engineered polypeptide described herein converts A1 to A2. In some embodiments, the engineered polypeptide can be used in a process of preparing the compound of formula A2 in an enantiomeric excess.

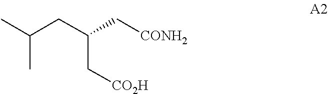

In these embodiments, said process comprises, under suitable reaction conditions, the compound shown in structural formula A1

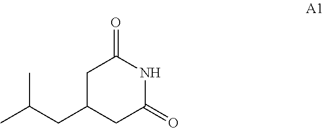

is contacted with the engineered polypeptide disclosed herein.

In some embodiments of the above process, the compound of Formula A2 is produced in an enantiomeric excess of at least 97%, 98%, 99% or more.

Specific embodiments of the engineered hydantoinase polypeptide for use in the process are provided further in the detailed description. Engineered polypeptides applicable in the above process may comprise amino acid sequences selected from SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270 272, 274, 276, 278, 280, 282, 284, 286, and may also comprise the amino acid sequence having at least 90%, 91%, 92%, 93%, 94%, 95%, 96%, 97%, 98%, or 99% sequence identity to any one of the reference amino acid sequences selected from SEQ ID NO: 4, 6, 8, 10, 12, 14, 16, 18, 20, 22, 24, 26, 28, 30, 32, 34, 36, 38, 40, 42, 44, 46, 48, 50, 52, 54, 56 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286.

As described herein and exemplified in the examples, the present disclosure contemplates a range of suitable reaction conditions that may be used in the process herein, including but not limited to pH, temperature, buffer, solvent system, substrate loading, polypeptide loading, and reaction time. Additional suitable reaction conditions for performing methods for enzymatically converting substrate compounds to product compounds using the engineered hydantoinase polypeptides described herein may be readily optimized by routine experimentation, which including but not limited to that the engineered polypeptide is contacted with the substrate compound under experimental reaction conditions of varying concentration, pH, temperature, solvent conditions, and the product compound is detected, for example, using the methods described in the Examples provided herein.

As described above, engineered polypeptides having hydantoinase activity for use in the process of the present disclosure generally comprises amino acid sequences that have at least 90%, 91%, 92%, 93%, 94%, 95%, 96%, 97%, 98%, or 99% sequence identity to any one of the reference amino acid sequences selected from SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262 264, 266, 268, 270, 272, 274, 276, 278, 280, 282, 284, 286.

The substrate compounds in the reaction mixture can be varied, taking into consideration of, for example, the amount of the desired product compound, the effect of the substrate concentration on the enzyme activity, the stability of the enzyme under the reaction conditions, and the percent conversion of substrate to product. In some embodiments of the process, the suitable reaction conditions include at least about 1 g/L, at least about 5 g/L, at least about 10 g/L, at least about 15 g/L, at least about 20 g/L, at least about 30 g/L, at least about 50 g/L, at least about 75 g/L, at least about 100 g/L, at least about 150 g/L, at least about 200 g/L, or even higher loadings of substrate A1. The values of the substrate loadings provided herein are based on the molecular weight of compound A1, however it is also anticipated that the equivalent molar amounts of various hydrates and salts of compound A1 may also be used in the process.

In embodiments of the reaction process, the reaction conditions may include a suitable pH. As described above, the desired pH or desired pH range may be maintained by the use of an acid or base, a suitable buffer, or a combination of buffering and addition of an acid or base. The pH of the reaction mixture may be controlled before and/or during the reaction process. In some embodiments, suitable reaction conditions include a solution pH of about 6 to about 8.5. In some embodiments, the reaction conditions include a solution pH of about 6, 6.5, 7, 7.5, 8, or 8.5.

In embodiments of the reaction processes herein, suitable temperatures may be used for the reaction conditions, taking into consideration of, for example, the increase in reaction rate at higher temperatures, the activity of the enzyme for sufficient duration of the reaction. Accordingly, in some embodiments, suitable reaction conditions include a temperature of about 10° C. to about 60° C., about 25° C. to about 50° C., about 25° C. to about 40° C., or about 25° C. to about 30° C. In some embodiments, a suitable reaction temperature comprises a temperature of about 25° C., 30° C., 35° C., 40° C., 45° C., 50° C., 55° C., or 60° C. In some embodiments, the temperature during the enzymatic reaction may be maintained at a certain temperature throughout the reaction. In some embodiments, the temperature during the enzymatic reaction may be adjusted over a temperature profile during the course of the reaction.

The processes of using the engineered hydantoinase are generally carried out in water or solvents. Suitable solvents include aqueous buffer solutions, organic solvents, and/or co-solvent systems, which generally include aqueous solvents and organic solvents. The aqueous solution (water or aqueous co-solvent system) may be pH-buffered or unbuffered. In some embodiments, the processes of using an engineered polypeptide are generally carried out in an aqueous co-solvent system comprising an organic solvent (e.g., methanol, ethanol, propanol, isopropyl alcohol (IPA)), dimethyl sulfoxide (DMSO), dimethyl formamide (DMF), isopropyl acetate, ethyl acetate, butyl acetate, 1-octanol, heptane, octane, methyl tert-butyl ether (MTBE), toluene, etc.), ionic liquids (e.g., 1-ethyl 4-methylimidazole tetrafluoroborate, 1-butyl-3-methylimidazole tetrafluoroborate, 1-butyl-3-methylimidazole hexafluorophosphate, etc.). The organic solvent component of the aqueous co-solvent system may be miscible with the aqueous component, providing a single liquid phase, or may be partially miscible or immiscible with the aqueous component, providing two liquid phases. The carbon dioxide generated during the hydrolysis reaction may cause foam formation, and antifoam agents may be added as appropriate. Exemplary aqueous co-solvent systems comprise water and one or more organic solvents. In general, the organic solvent component of the aqueous co-solvent system is selected such that it does not completely inactivate the hydantoinase. Suitable co-solvent systems can be readily identified by measuring the enzymatic activity of a particular engineered hydantoinase with a defined substrate of interest in the candidate solvent system, utilizing enzymatic activity assay such as those described herein.

Suitable reaction conditions may include combinations of reaction parameters that provide for the biocatalytic conversion of the substrate compound to its corresponding product compound. Accordingly, in some embodiments of the process, the combination of reaction parameters includes (a) a loading of about 1 g/L to 400 g/L of substrate A1; (b) an engineered polypeptide concentration of about 0.1 g/L to 50 g/L; (c) a pH of about 6.0 to 8.5; and (d) a temperature of about 10° C. to 60° C.

In some embodiments, the process described above comprises contacting >10 g/L of A1 substrate with the engineered polypeptide described herein at a temperature of about 30° C. to about 50° C., a pH of 6.0 to 8.0; and within 24 hours, at least 70%, 80%, 90%, 95%, or more of the substrate A1 is converted to product A2, and product A2 is produced in an enantiomeric excess of at least 97%, 98%, 99% or more. In some embodiments, the hydantoinase polypeptide capable of the above reaction comprises an amino acid sequence corresponding to the even numbered sequences of SEQ ID NO:4-286.

Exemplary reaction conditions include the assay conditions provided in Examples 12-22.

In carrying out the enzyme-catalyzed reactions described herein, the engineered polypeptide may be added to the reaction mixture in the form of a partially purified or purified enzyme, a heat-treated enzyme solution, whole cells transformed with the gene encoding the engineered polypeptide, and/or as cell extracts and/or lysates of such cells. Whole cells transformed with the genes encoding the engineered polypeptides, or cell extracts thereof, lysates thereof, and isolated enzymes can be used in a variety of different forms, including solid (e.g., lyophilized, spray dried, etc.) or semi-solid (e.g., a crude pastes). The cell extracts or cell lysates may be partially purified by precipitation (e.g., ammonium sulfate, polyethyleneimine, heat treatment, or the like), followed by a desalting procedure (e.g., ultrafiltration, dialysis, and the like) priorto lyophilization. Any of the enzyme preparations can be stabilized by crosslinking using known crosslinking agents, such as glutaraldehyde, or immobilization to a solid phase material (such as a resin).

In some embodiments of the enzyme-catalyzed reactions described herein, the reactions are carried out under suitable reaction conditions as described herein, wherein the engineered polypeptide is immobilized to a solid support. Solid supports useful for immobilizing the engineered polypeptide for carrying out the reaction include but are not limited to beads or resins such as polymethacrylates with epoxy functional groups, polymethacrylates with amino epoxy functional groups, polymethacrylates, styrene/DVB copolymer or polymethacrylates with octadecyl functional groups. Exemplary solid supports include, but are not limited to, chitosan beads, Eupergit C, and SEPABEADs (Mitsubishi), including the following different types of SEPABEAD: EC-EP, EC-HFA/S, EXA252, EXE119 and EXE120.

In some embodiments, wherein the engineered polypeptide may be expressed in the form of a secreted polypeptide, a culture medium containing the secreted polypeptide may be used in the process herein.

In some embodiments, the solid reactants (e.g., enzymes, salts, etc.) may be provided to the reaction in a variety of different forms, including powders (e.g., lyophilized, spray dried, etc.), solutions, emulsions, suspensions and the like. The reactants can be readily lyophilized or spray dried using methods and instrumentation known to one skilled in the art. For example, the protein solution can be frozen at −80° C. in small aliquots, and then added to the pre-chilled lyophilization chamber, followed by the application of a vacuum.

In some embodiments, there are various options for the order or manner in which the reactants are added. The reactants may be added together to the solvent at the same time (e.g., monophasic solvent, a biphasic aqueous co-solvent system, etc.), or alternatively, some reactants may be added first and others may be added flow-through or in batch intervals.

Different features and embodiments of the present disclosure are exemplified in the following representative embodiments, which are intended to be illustrative and not restrictive.

### EXAMPLES

The following examples further illustrate the present invention, but the present invention is not limited thereto. In the following examples, experimental methods with conditions not specified, were conducted at the commonly used conditions or according to the supplier's suggestion.

### Example 1: Gene Cloning and Construction of Expression Vectors

The amino acid sequence of the wild-type hydantoinase from Pseudomonas fluorescens can be retrieved from NCBI (GenBank: KF268426.1), and the corresponding nucleic acids were then synthesized by a vendor using conventional techniques in the art and cloned into the expression vector pACYC-Duet-1 (Novagen). The recombinant expression plasmid was transformed into E. coli BL21 (DE3) competent cells under the conditions of 42° C. and thermal shock for 90 seconds. The transformation solution was plated on LB agar plates containing chloramphenicol which was then incubated overnight at 37° C. Recombinant transformants were obtained.

### Example 2: Construction of Hydantoinase Mutant Library

All the reagents used here are commercial reagents, Quikchange kit (supplier: Agilent) was preferably used. The sequence design of the mutagenesis primers was performed according to the instructions of the kit.

The PCR system was: 10×buffer 2.5 μL, dNTP mix 1 μL, primer Oligomix 2 μL (5 μM), plasmid template 2.5 μL (50ng/μl), high fidelity enzyme 1 μL, ddH₂O 16 μL.

The PCR amplification steps were: (1) 95° C., pre-denaturation 1 min; (2) 95° C., denaturation 1 min; (3) 55° C., annealing 1 min; (4) 65° C., extension 6 min; steps (2)-(4) repeated 29 times; (5) 65° C., extension was continued for 5 min and cooled to 4° C. 2 μl of Dpnl (Kit) was added to the PCR product, and digestion at 37° C. for 2 h. The product was transformed to E. coli BL21 (DE3) competent cells and plated on LB agar plates containing chloramphenicol, and incubate upside down at 37° C. overnight to obtain library colonies.

### Example 3: Expression of Mutant Library and Preparation of Enzyme Solution for Screening

Mutant colonies were picked from the LB agar plates, inoculated into LB medium (containing chloramphenicol) in a 96-well shallow plate and cultured overnight at 30° C. When OD₆₀₀ of deep-well culture reached 2˜3, 20 μl of the above culture was used to inoculate TB medium (400 μL TB medium per well, including chloramphenicol) in a deep-well plate and cultured at 30° C. When OD₆₀₀ of deep-well culture reached 0.6 0.8, and IPTG was added to induce expression at a final concentration of 1 mM, and the expression undertook at 30° C. overnight (18-20 h). Once the overnight expression was done, the culture was centrifuged, and the supernatant of the solution was removed to obtain wet cell pellets. The cell lylsis buffer (1 g/L lysozyme, 0.5 g/L PMBS, dissolved in PBS buffer, pH7) was added to the cell pellets and shaken for 1 h to break the cells to obtain the lysate. The lysate was centrifuged and the supernatant was transferred to a new deep-well plate to obtain an enzyme solution that would be used for the screening assays.

### Example 4: Expression of Engineered Polypeptide

A single colony of E. coli BL21 (DE3) with the expression plasmid of target engineered polypeptide was inoculated into a 250 mL conical flask containing 50 mL LB medium with 30 μg/mL chloramphenicol and cultured in a shaking incubator overnight at 30° C. When the OD₆₀₀ of the culture medium reached 2, the culture was subcultured into a 1000 mL conical flask containing 250 mL of TB medium at 5% (v/v) inoculum and incubated at 30° cin a shaking incubator. When the OD₆₀₀ of the TB culture medium reached 0.6, IPTG was added to induce the expression of hydantoinase at a final concentration of 1 mM. After expression of 20 h, the culture was centrifuged (8000 rpm, 10 min), and the supernatant was discarded after centrifugation, and the cells were collected to obtain wet cells. The wet cells were used directly in the preparation of enzyme solution or could be stored frozen at −20° C. until use.

The wet cells were resuspended in PBS buffer, sonicated in an ice bath, and the supernatant was collected by centrifugation to obtain the enzyme solution containing the engineered polypeptide.

### Example 5: Quantification of Hydantoinase Polypeptides in Enzyme Solution Samples

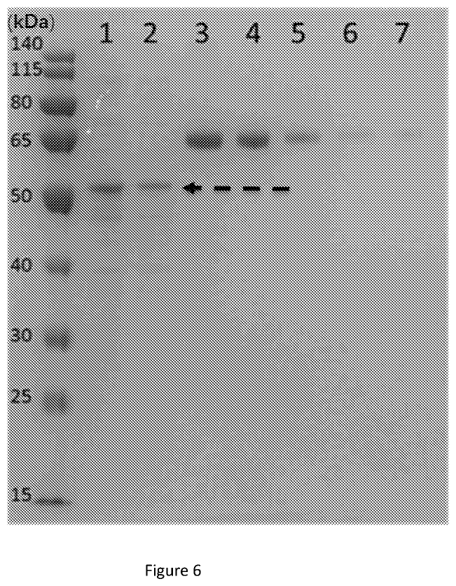

According to the method of Example 4, the enzyme solution of SEQ ID NO: 2 was prepared, diluted 100 times (sample 1) and 200 times (sample 2), and analyzed by electrophoresis together with different concentrations of BCA protein standard samples (Easy II Protein Quantitative Kit, brand: Transgen). The grayscale analysis of protein bands on the electrophoresis gel image were performed by computer software, and a standard curve of the grayscale values of BCA bands (samples 3-7 in FIG. 6) and BCA concentration was obtained. The concentration of hydantoinase polypeptide in the enzyme solution sample can be obtained by fitting the grayscale value of the target band of hydantoinase enzyme solution (shown by the dashed arrow in FIG. 6) into the equation of the standard curve.

| | Electrophoresis sample number | | | | | | |
|---|---|---|---|---|---|---|---|
| | 1 | 2 | 3 | 4 | 5 | 6 | 7 |
| Electrophoresis | enzyme | enzyme | BCA | BCA | BCA | BCA | BCA |
| samples | solution | solution | | | | | |
| | 1 | 2 | | | | | |
| protein | 42.9 | 23.9 | 100 | 50 | 25 | 12.5 | 6.25 |
| concentration | | | | | | | |
| (μg/mL) | | | | | | | |

### Example 6: High Throughput Analysis Method for Measuring Conversion of 96-Well Plate Samples

HPLC analysis method: the column was Gemini C18 250 mm*4.6 mm*5 um, the mobile phase was 70% 0.4% HCLO4:30% ACN, the flow rate was 1 mL/min, the column temperature was 40° C., the detection wavelength was 210 nm, the solvent was 50% ACN, the injection volume was 10 uL, where the retention time of (R)-(−)-3-(carbamoylmethyl)-5-methylhexanoic acid was 5.030 min and that of 3-isobutylglutarimide was 11.188 min.

### Example 7: Chiral Analysis Method

Sample derivatization process: 1 mL of reaction solution was taken, potassium carbonate and 2-bromoacetophenone were weighed in the ratio of product: potassium carbonate: 2-bromoacetophenone (mass ratio)=5:3:1, 1 mL of acetonitrile was added and mixed with 1 mL of reaction solution and shaken at 1500 rpm for 15 min, 3 mL of ethyl acetate was added and shaken at 1500 rpm for 15 min. The ethyl acetate layer was taken after centrifugation and lyophilized, dissolved with 50% ACN and then detected by HPLC.

HPLC method: The column was CHIRALPAK AD-RH 4.6*150 mm*5 um, the mobile phase was 50% water (pH adjusted to 2.50 by phosphoric acid): 50% ACN, the flow rate was 0.5 ml/min, the column temperature was 30° C., the detection wavelength was 210 nm, the injection volume was 10 ul. The retention time of (R)-(−)-3-(carbamoylmethyl)-5-methylhexanoic acid (R-CMH) was 15.2 min, and the retention time of (S)-(−)-3-(carbamoylmethyl)-5-methylhexanoic acid (S-CMH) was 13.2 min.

ee={[R-CMH]−[S-CMH]}/{[R-CMH]+[S-CMH]}.

### Example 8: Screening Assay Reactions for Catalytic Activity in the First Stage of Directed Evolution

Referring to the method of Example 3, the enzyme solution of pH 7.0 was prepared and immediately used to perform the screening reaction.

In a 96-well plate, the enzyme solution was mixed with the substrate stock solution (prepared by dissolving substrate A1 in DMSO) to make the final concentration of each component in the reaction system as [substrate 2 g/L, DMSO 10%, enzyme 10 g/L, 0.05 M PBS], and the plate was placed in a shaker at 250 rpm and 30° C. for 22 h. After the reaction, 200 μL of pure acetonitrile was added to each well to quench the reaction; the plate was shaken for 30 min (800 rpm), then centrifuged (4000 rpm, 10 min), and the supernatant after centrifugation was taken and analyzed by HPLC according to the method of Example 6. For each sample, the conversion of A1 to A2 was calculated, and the ee value of product A2 was determined according to the method of Example 7.

### Example 9: Screening Assay Reactions for pH Stability in the Second Stage of Directed Evolution

Referring to the method of Example 3, the enzyme solution of pH 6.3 was prepared and shaken at room temperature (20° C.-25° C.) for 23 hours, and then PBS buffer was added to adjust the pH of the enzyme solution to 7.0 for the screening reaction.

In a 96-well plate, the pretreated enzyme solution was mixed with the substrate stock solution (prepared by dissolving substrate A1 in DMSO) to make the final concentration of each component in the reaction system as [substrate 2 g/L, DMSO 10%, enzyme 3 g/L, 0.05M PBS], and the plate was placed in a shaker at 250 rpm and 30° C. for 22 hours. After the reaction, 200 μL of pure acetonitrile was added to each well to quench the reaction; the plate was shaken for 30 min (800 rpm), then centrifuged (4000 rpm, 10 min), and the supernatant after centrifugation was taken and analyzed by HPLC according to the method of Example 6. For each sample, the conversion of A1 to A2 was calculated, and the ee value of product A2 was determined according to the method of Example 7.

### Example 10: Screening Assay Reaction for Product Tolerance in the Third Stage of Directed Evolution

Referring to the method of Example 3, the enzyme solution of pH 7.0 was prepared and the screening reaction was performed immediately.

In a 96-well plate, the enzyme solution was mixed with the substrate stock solution (prepared by dissolving substrate A1 in DMSO) and the product stock solution (prepared by dissolving product A2 in PBS buffer) to make the final concentration of each component in the reaction system as [substrate 2 g/L, product A2 50 g/L, DMSO 10%, enzyme 0.3 g/L, 0.05 M PBS], and the well plate was placed in a shaker at 250 rpm, 30° C. for 22 hours. After the reaction, 200 μL of pure acetonitrile was added to each well to quench the reaction, the plate was shaken for 30 min (800 rpm), then centrifuged (4000 rpm, 10 min), and the supernatant after centrifugation was taken and analyzed by HPLC according to the method of Example 6 and the conversion of A1 to A2 was calculated.

### Example 11: Screening Assay Reaction for Thermostability in the Third Stage of Directed Evolution

Referring to the method of Example 3, the enzyme solution of pH 7.0 was prepared and shaken at 50° C. for 23 hours, and then the screening reaction was performed.

In a 96-well plate, the enzyme solution was mixed with the substrate stock solution (made by dissolving substrate A1 in DMSO) to make the final concentration of each component in the reaction system as [substrate A1 2 g/L, DMSO 10%, enzyme 0.3 g/L, 0.05M PBS], and the well plate was placed in a shaker at 250 rpm and 30° C. for 22 hours. After the reaction, 200 μL of pure acetonitrile was added to each well to quench the reaction, the plate was shaken for 30 min (800 rpm), then centrifuged (4000 rpm, 10 min), and the supernatant after centrifugation was taken and analyzed by HPLC according to the method of Example 6 and the conversion of A1 to A2 was calculated.

### Example 12: Method for Measuring the Conversion in a 5 mL Reaction of the Engineered Polypeptides from the First Stage of Directed Evolution

250 mg of wet cells expressing SEQ ID NO: 8 and 50 mg of substrate A1 were charged into a reaction flask with a total volume of 30 mL, and finally PBS buffer (0.1 M, pH 7.0) was added to make the total reaction volume 5.0 mL, and the concentration of each component in the reaction system was [50 g/L of wet cells and 10 g/L of substrate A1]. The reaction was placed on a magnetic stirrer set at 400 rpm and 30° C. After 24 h of reaction, the reaction was quenched by adding 5 mL of acetonitrile and mixing for 30 min. The quenched reaction sample was transferred to a 2 mL centrifuge tube and then centrifuged (13000 rpm, 3 min), and the supernatant after centrifugation was taken and analyzed by HPLC according to the method of Example 6 and the method of Example 7.

### Example 13: Method for Measuring the Conversion in a 5 mL Reaction of the Engineered Polypeptides from the Second Stage of Directed Evolution

30 mg of wet cells expressing SEQ ID NO: 50 and 50 mg of substrate A1 were charged into a reaction flask with a total volume of 30 mL, and finally PBS buffer (0.1 M, pH 7.0) was added to make the total reaction volume 5.0 mL, and the concentration of each component in the reaction system was [6 g/L of wet cells and 10 g/L of substrate A1]. The reaction was placed a magnetic stirrer set at 400 rpm and 30° C. After 24 h of reaction, the reaction was quenched by adding 5 mL of acetonitrile to the flask and mixing for 30 min. The quenched solution sample was transferred to a 2 mL centrifuge tube and then centrifuged (13000 rpm, 3 min), and the supernatant after centrifugation was taken and analyzed by HPLC according to the method of Example 6 and the method of Example 7.

### Example 14: Method for Measuring the Conversion in a 5 mL Reaction of the Engineered Polypeptides from the Third Stage of Directed Evolution

5 mg of wet cells expressing SEQ ID NO: 184 and 50 mg of substrate A1 were charged into a reaction flask with a total volume of 30 mL, and finally PBS buffer (0.1 M, pH 7.0) was added to make the total reaction volume 5.0 mL, and the concentration of each component in the reaction system was [1 g/L of wet cell and 10 g/L of substrate A1]. The reaction was placed on a magnetic stirrer set at 400 rpm and 40° C. After 24 h of reaction, the reaction was quenched by adding 5 mL of acetonitrile to the flask and mixing for 30 min. The quenched solution was transferred to a 2 mL centrifuge tube and then centrifuged (13000 rpm, 3 min), and the supernatant after centrifugation was taken and analyzed by HPLC according to the method of Example 6 and the method of Example 7.

### Example 15: Process for the Synthesis of Pregabalin Intermediate Catalyzed by Engineered Hydantoinase Polypeptide SEQ ID No: 10

100 mL of 0.05 M PBS pH 7.0 buffer was charged in a reaction vessel with a total volume of 500 mL, then 50 mL of enzyme solution (SEQ ID No: 10) was charged, the water bath was used to maintain the temperature at 30° C. The reaction was stirred at 200 rpm, and finally 3 g of substrate A1 (3-isobutylglutarimide) was charged to start the reaction. During the reaction, the pH was adjusted with ammonia and maintained at 7.0±0.2, and the reaction was terminated after 20 h. The conversion was 71% by sampling.

The reaction solution was filtered with diatomaceous earth, and the filtrate was concentrated to about 100 mL, then the pH of the concentrated filtrate was adjusted to 3.0 by adding hydrochloric acid dropwise to allow the crystallization of product A2. After stirring for 30 min, the wet crude product was filtered, dried and weighed. 2.2 g of crude product A2 was obtained, ee=99.8%.

### Example 16: Process for the Synthesis of Pregabalin Intermediate Catalyzed by Engineered Hydantoinase Polypeptide SEQ ID No: 24

100 mL of 0.05M PBS pH7.0 buffer was charged in a reaction vessel with a total volume of 500 mL, then 50 mL of enzyme solution (SEQ ID No: 24) was charged, the water bath was used to maintain the temperature at 30° C. The reaction was stirred at 200 rpm, and finally 3 g of substrate A1 (3-isobutylglutarimide) was charged to start the reaction. During the reaction, the pH was adjusted with ammonia and maintained at 7.0±0.2, and the reaction was terminated after 20 h. The conversion was 73% by sampling.

The reaction solution was filtered with diatomaceous earth, and the filtrate was concentrated to about 100 mL, then the pH of the concentrated filtrate was adjusted to 3.0 by adding hydrochloric acid dropwise to allow the crystallization of product A2. After stirring for 30 min, the wet crude product was filtered, dried and weighed. 2.3 g of crude product A2 was obtained, ee=99.7%.

### Example 17: Process for the Synthesis of Pregabalin Intermediate Catalyzed by Engineered Hydantoinase Polypeptide SEQ ID No: 52

140 mL of 0.05M PBS pH7.0 buffer was charged in a reaction vessel with a total volume of 500 mL, then 10 mL of enzyme solution (SEQ ID No: 52) was charged, the water bath was used to maintain the temperature at 35° C. The reaction was stirred at 200 rpm, and finally 10 g of substrate A1 (3-isobutylglutarimide) was charged to start the reaction. During the reaction, the pH was adjusted with ammonia and maintained at 7.0±0.2, and the reaction was terminated after 20 h. The conversion rate was 95% by sampling.

The reaction solution was filtered with diatomaceous earth, and the filtrate was concentrated to about 100 mL, then the pH of the concentrated filtrate was adjusted to 3.0 by adding hydrochloric acid dropwise to allow the crystallization of product A2. After stirring for 30 min, the wet crude product was filtered, dried and weighed. 10.1 g of crude product A2 was obtained, ee ≥99.6%.

### Example 18: Process for the Synthesis of Pregabalin Intermediate Catalyzed by Engineered Hydantoinase Polypeptide SEQ ID No: 162

140 mL of 0.05M PBS pH7.0 buffer was charged in a reaction vessel with a total volume of 500 mL, then 10 mL of enzyme solution (SEQ ID No: 162) was charged, the water bath was used to maintain the temperature at 35° C. The reaction was stirred at 200 rpm, and finally 10 g of substrate A1 (3-isobutylglutarimide) was charged to start the reaction. During the reaction, the pH was adjusted with ammonia and maintained at 7.0±0.2, and the reaction was terminated after 20 h. The conversion was 96% by sampling.

The reaction solution was filtered with diatomaceous earth, and the filtrate was concentrated to about 100 mL, then the pH of the concentrated filtrate was adjusted to 3.0 by adding hydrochloric acid dropwise to allow the crystallization of product A2. After stirring for 30 min, the wet crude product was filtered, dried and weighed. 10.4 g of crude product A2 was obtained, ee=99.5%.

### Example 19: Process for the Synthesis of Pregabalin Intermediate Catalyzed by Engineered Hydantoinase Polypeptide SEQ ID No: 184

145 mL of 0.05M PBS pH7.0 buffer was charged in a reaction vessel with a total volume of 500 mL, then 5 mL of enzyme solution (SEQ ID No: 184) was charged, the water bath was used to maintain the temperature at 45° C. The reaction was stirred at 200 rpm, and finally 30 g of substrate A1 (3-isobutylglutarimide) was charged to start the reaction. During the reaction, the pH was adjusted with ammonia and maintained at 7.0±0.2, and the reaction was terminated after 20 h. The conversion was 98% by sampling.

The reaction solution was filtered with diatomaceous earth, and the filtrate was concentrated to about 100 mL, then the pH of the concentrated filtrate was adjusted to 3.0 by adding hydrochloric acid dropwise to allow the crystallization of product A2. After stirring for 30 min, the wet crude product was filtered, dried and weighed. 32.1 g of crude product A2 was obtained, ee=99.7%.

### Example 20: Process for the Synthesis of Pregabalin Intermediate Catalyzed by Engineered Hydantoinase Polypeptide SEQ ID No: 264

145 mL of 0.05M PBS pH7.0 buffer was charged in a reaction vessel with a total volume of 500 mL, then 5 mL of enzyme solution (SEQ ID No: 264) was charged, the water bath was used to maintain the temperature at 45° C. The reaction was stirred at 200 rpm, and finally 20 g of substrate A1 (3-isobutylglutarimide) was charged to start the reaction. During the reaction, the pH was adjusted with ammonia and maintained at 7.0±0.2, and the reaction was terminated after 20 h. The conversion was 72% by sampling.

The reaction solution was filtered with diatomaceous earth, and the filtrate was concentrated to about 100 mL, then the pH of the concentrated filtrate was adjusted to 3.0 by adding hydrochloric acid dropwise to allow the crystallization of product A2. After stirring for 30 min, the wet crude product was filtered, dried and weighed. 15.8 g of crude product A2 was obtained, ee=99.8%.

### Example 21: Process for the Synthesis of Pregabalin Intermediate Catalyzed by Engineered Hydantoinase Polypeptide SEQ ID No: 286

145 mL of 0.05M PBS pH7.0 buffer was charged in a reaction vessel with a total volume of 500 mL, then 5 mL of enzyme solution (SEQ ID No: 286) was charged, the water bath was used to maintain the temperature at 45° C. The reaction was stirred at 200 rpm, and finally 36 g of substrate A1 (3-isobutylglutarimide) was charged to start the reaction. During the reaction, the pH was adjusted with ammonia and maintained at 7.0±0.2, and the reaction was terminated after 20 h. The conversion was 96% by sampling.

The reaction solution was filtered with diatomaceous earth, and the filtrate was concentrated to about 100 mL, then the pH of the concentrated filtrate was adjusted to 3.0 by adding hydrochloric acid dropwise to allow the crystallization of product A2. After stirring for 30 min, the wet crude product was filtered, dried and weighed. 37.9 g of crude product A2 was obtained, ee=99.8%.

### Example 22: Process for the Synthesis of D-p-Hydroxyphenylglycine Catalyzed by Engineered Hydantoinase Polypeptide SEQ ID No: 214

The following is a representative process at a 5 mL reaction volume. 70 μL of enzyme solution of SEQ ID NO:214 was charged in a reaction flask with a total volume of 30 mL, 50 mg of p-hydroxyphenylhydantoin was then charged, and finally 5 mL of phosphate buffer (0.1 M, pH 7.5) was added to make the concentration of each component in the reaction system as [14 mL/L of enzyme solution of SEQ ID NO:214, 10 g/L of p-hydroxyphenylhydantoin]. The reaction flask was placed on an IKA magnetic stirrer set at 400 rpm and 40° C. to start the reaction. After 1 hour of reaction, 5 mL of acetonitrile was added to quench the reaction. Concentrated hydrochloric acid was added to the quenched reaction to a final concentration of 2 mmol/L, then 27 mg of sodium bisulfite was added to the reaction flask, placed on a magnetic stirrer set at 400 rpm and 50° C. to start hydrolysis. After 3 h, 5 mL of 0.1% glacial acetic acid was added to the reaction flask, and the reaction solution was centrifuged (13000 rpm, 3 min). The supernatant of the centrifuged sample was analyzed by HPLC. The conversion was measured as 42.3%.

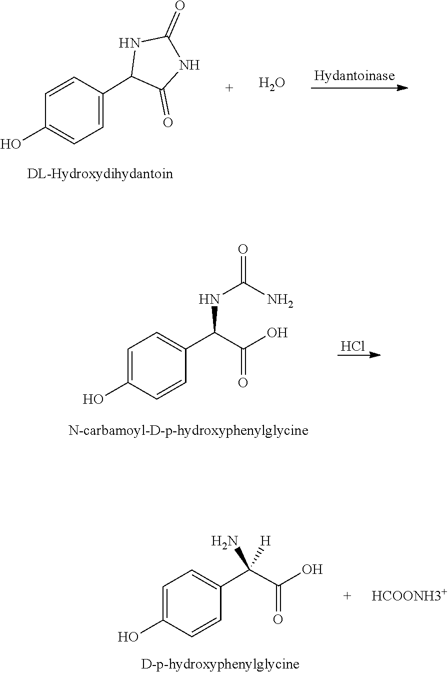 [SMILES: O=C1NC(=O)C(c2ccc(O)cc2)N1 ; O ; NC(=O)N[C@@H](C(=O)O)c1ccc(O)cc1 ; Cl ; N[C@@H](C(=O)O)c1ccc(O)cc1]

It should be understood that after reading the above contents of the present invention, those skilled in the art may make various modifications or changes to the present invention. And these equivalent forms also fall within the scope of the appended claims of the present invention.

1. An engineered hydantoinase polypeptide that catalyzes the asymmetric hydrolysis of 3-isobutylglutarimide to generate (R)-(−)-3-(carbamoylmethyl)-5-methylhexanoic acid with an entantiomeric excess (ee) value of at least 97%, wherein said polypeptide comprises an X64 substitution and a at least 90% sequence identity to reference sequence SEQ ID NO.2, wherein the amino acid residue at residue position X64 is selected from the group consisting of I, T, S and A.

2. The engineered hydantoinase polypeptide according to claim 1, wherein reaction conditions of said asymmetric hydrolysis comprises a load of about 1 g/L-400 g/L 3-isobutylglutarimide, a load of 0.1 g/L to 50 g/L engineered polypeptide, a pH of 6.0 to 8.5, and a temperature of 10-60° C.

3. The polypeptide according to claim 1, wherein the amino acid sequence of the polypeptide is selected from the group consisting of SEQ ID No 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, and 286.

4. A polypeptide immobilized on a solid material by a chemical bonds or a physical adsorption method, wherein the polypeptide comprises the engineered hydantoinase polypeptide of claim 1.

5. A polynucleotide encoding the polypeptide of claim 1.

6. The polynucleotide of claim 5, wherein said polynucleotide comprises a nucleotide sequence selected from the group consisting of SEQ ID No: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, and 285.

7. An expression vector comprising the polynucleotide of claim 5.

8. The expression vector of claim 7, wherein said vector comprises a plasmid, a cosmid, a bacteriophage or a viral vector.

9. A host cell comprising the expression vector of claim 8.

10. A method of preparing a hydantoinase polypeptide, wherein said method comprises the steps of culturing the host cell of claim 9 and obtaining a hydantoinase polypeptide from the culture.

11. A hydantoinase catalyst obtainable by the method of claim 10, wherein said hydantoinase catalyst comprises cells or culture fluid containing hydantoinase polypeptides, or an article processed therewith, wherein said article comprises an extract obtained from the host cell, an isolated product obtained by isolating or purifying a hydantoinase from the extract, an immobilized product obtained by immobilizing said host cell, an extract said immobilized product, or isolated product of the extract.

12. A process of preparing a compound of formula (I): 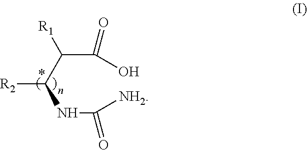
wherein the compound of structural formula (I) has the indicated stereochemical configuration at the chiral center marked with * and is present in an enantiomeric excess over the other enantiomer, further wherein,
n=0 or 1;
R₁ and R₂ are independently of each other selected from (a) H, (b) optionally substituted or unsubstituted aryl or heteroaryl, (c) straight or branched and optionally substituted or unsubstituted C₁-C₄ alkyl, (d) straight or branched and optionally substituted or unsubstituted C₁-C₄ alkenyl, (e) optionally substituted or unsubstituted cycloalkyl, (f) —OR′, (g) —NH₂, (h) —NR′R′, (i) —SR′, (j) —CO₂R′, or (k) —C(O)R′; and
each R′ is independently selected from —H or (C₁-C₄) hydrocarbon groups; wherein said process comprises the step of contacting the engineered hydantoinase polypeptide of claim 1 with a hydantoin-derived substrate of formula (II), 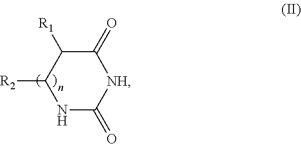
wherein the definitions for variables n, R₁, R₂ in said structural formula (II) are the same as in structural formula (I).

13. A process of preparing a compound of formula (III): 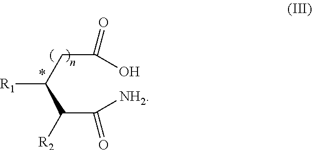
wherein the compound of structural formula (III) has the indicated stereochemical configuration at the chiral center marked with *; the compound of said structural formula (III) is in an enantiomeric excess over the other enantiomer, further wherein,
n=0 or 1; and
R₁, R₂ are independently of each other selected from the group consisting of (a) H, (b) straight or branched and optionally substituted or unsubstituted C₁-C₄ alkyl, and (c) optionally substituted or unsubstituted C₆H₆;
provided that when n=0, R₁ and R₂ may together form a monocyclic or polycyclic ring structure group selected from the group consisting of (a) optionally substituted or unsubstituted aryl groups and (b) optionally substituted or unsubstituted heteroaryl groups;
wherein said process comprises the step of contacting the engineered hydantoinase polyeptide of claim 1 with an acyl imide derived substrate of formula (IV), 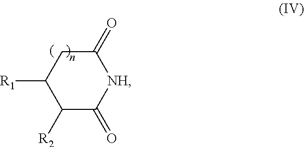
wherein the definitions for variables ef-n, R₁, R₂ in said structural formula (IV) are the same as in structural formula (III).

14. A process of preparing a compound of D-p-hydroxyphenylglycine, said process comprising the steps of: (1) converting substrate DL-p-hydroxyphenylhydantoin depicted below 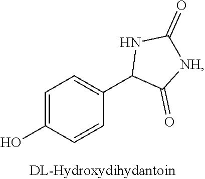 [SMILES: O=C1NC(=O)C(c2ccc(O)cc2)N1]
into N-carbamyl-D-p-hydroxyphenylglycine depicted below in the presence of the engineered hydantoinase polypeptide of claim 1, 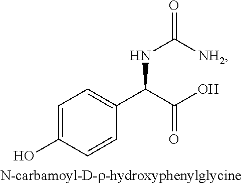 [SMILES: NC(=O)N[C@@H](C(=O)O)c1ccc(O)cc1]
and (2) further converting said N-carbamyl-D-p-hydroxyphenylglycine to D-p-hydroxyphenylglycine in the presence of hydrochloric acid

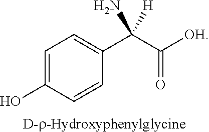 [SMILES: N[C@@H](C(=O)O)c1ccc(O)cc1]

15. A process of preparing the compound of formula A2 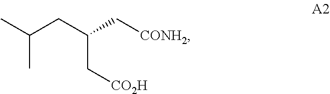
said process comprising the step of contacting, under suitable reaction conditions, the compound of formula A1 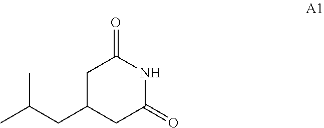
with the engineered hydantoinase polypeptide of claim 1.

16. The process of claim 12, wherein the product is produced in an enantiomeric excess of at least 97%, 98%, 99% or more.

17. The process of claim 12, wherein said step of contacting the engineered hydantoinase polypeptide with said hydantoin-derived substrate occurs in a reaction solvent selected from the group consisting of water, methanol, ethanol, propanol, isopropanol, dimethyl sulfoxide, dimethylformamide, isopropyl acetate ester, ethyl acetate, butyl acetate, 1-octanol, heptane, octane, methyl tert-butyl ether (MTBE), and toluene.

18. The process of claim 12, wherein said reaction conditions under which said hydantoin-derived substrate is contacted with said comprise a temperature of 10° C. to 60° C.

19. The process of claim 12, wherein said reaction conditions under which said hydantoin-derived substrate is contacted with said comprise pH of 6.0 to pH 8.5.

20. The process of claim 12, wherein said hydantoin-derived substrate is present at a loading of 1 g/L to 400 g/L.
